(19) United States
(12) Patent Application Publication
(10) Pub. No.: US 2026/0306675 A1
(43) Pub. Date: Oct. 8, 2026

(54) EPIGENETIC REPRESSION PROTEIN THERAPY FOR RASOPATHIES

(71) Applicants: The Regents of the University of California, Oakland, CA (US); Battelle Memorial Institute, Columbus, OH (US)

(72) Inventors: David J. SEGAL, Davis, CA (US); Niraj PUNJYA, Davis, CA (US); Kenneth R. SIMS, Jr., Columbus, OH (US); Chelsi SNOW, Columbus, OH (US)

(73) Assignees: The Regents of the University of California, Oakland, CA (US); Battelle Memorial Institute, Columbus, OH (US)

(21) Appl. No.: 19/484,174

(22) PCT Filed: May 17, 2024
(86) PCT No.: PCT/US2024/029905

§ 371 Date: Nov. 13, 2025

Related U.S. Application Data
(60) Provisional application No. 63/503,091, filed on May 18, 2023.

(51) Int. Cl. A61K 48/00; A61P 43/00; C12N 9/10; C12N 9/22; C12N 15/11

(52) U.S. Cl. CPC A61K 48/0058; A61P 43/00; C12N 9/1007; C12N 9/226; C12N 15/111; C07K 2319/80; C12N 2310/20; C12N 2830/008; C12Y 201/01037

(57) ABSTRACT

The present disclosure provides a CRISPR-based epigenetic repression system downregulating MAP2K1 and MAP2K2 expression for the treatment of RASopathies, such as neurofibromatosis type 1 (NF1).

### CROSS-REFERENCE TO RELATED APPLICATIONS

This application claims priority to U.S. Provisional Application No. 63/503,091, filed May 18, 2023, the disclosure of which is herein incorporated by reference in its entirety for all purposes.

### BACKGROUND

RASopathies are a group of syndromes, also called conditions or disorders, caused by changes in genes that send signals across the Ras/mitogen-activated protein kinase (Ras/MAPK) pathway. Neurofibromatosis type 1 (NF1) is a kind of RASopathy that affects the nervous system, skin, and other organs. It is caused by mutations in the neurofibromin gene, which helps regulate cell growth and division. NF1 can cause a wide range of symptoms, including the development of tumors on or under the skin, freckling of the skin in certain patterns, and abnormalities in bone growth. It can also lead to learning disabilities, seizures, vision and hearing problems, and other neurological issues. Symptoms can vary widely between individuals and can range from mild to severe. There is no cure for NF1, but treatment focuses on managing symptoms and preventing complications. Treatment may involve surgery to remove tumors, medications to manage pain or other symptoms, and monitoring for complications such as high blood pressure or vision problems. An epigenetic therapy can provide an alternative way to address the disorder. The present disclosure satisfies this need and provides related advantages as well.

### SUMMARY

In one aspect, the present disclosure provides a recombinant polynucleotide comprising (a) a first nucleic acid segment encoding a fusion protein comprising an RNA-guided programmable DNA-binding domain fused to a repressor domain; and (b) a second nucleic acid segment encoding at least one guide RNA targeting a MAP2K gene. In some embodiments, the RNA-guided programmable DNA-binding domain comprises a nuclease-deficient RNA-guided DNA endonuclease. In some embodiments, the nuclease-deficient RNA-guided DNA endonuclease is dCas9.

In various embodiments, the repressor domain comprises a Krüppel-associated box (KRAB) domain, an mSin3 interaction domain (SID), a four concatenated mSin3 interaction domain (SID4X), a MAX-interacting protein 1 (MXI1), a chromo shadow domain, an EAR-repression domain (SRDX), eukaryotic release factor 1 (ERF1), eukaryotic release factor 3 (ERF3), tetracycline repressor, the lad repressor, Catharanthus roseus G-box binding factors 1 and 2, Drosophila Groucho, Tripartite motif-containing 28 (TRIM28), Nuclear receptor co-repressor 1, Nuclear receptor co-repressor 2, or any combination, fragment, or fusion thereof. In particular embodiments, the repressor domain comprises a Krüppel-associated box (KRAB) domain.

In some embodiments, the fusion protein further comprises an effector domain. In some embodiments, the effector domain comprises a DNMT3A domain, a DNMT3A with DNA Methyltransferase 3-Like protein (DNMT3A/3L) domain, a histone-lysine N-methyltransferase SUV39H1, a G9a, an enhancer of zeste homolog 2 (Ezh2), or any combination, fragment, or fusion thereof. In particular embodiments, the effector domain comprises a DNMT3A/3L domain. In certain embodiments, the fusion protein comprises, from N-terminus to C-terminus, an effector domain, an RNA-guided programmable DNA-binding domain, and a repressor domain.

As disclosed herein, the MAP2K gene can be a MAP2K1 gene or a MAP2K2 gene. In some embodiments, the second nucleic acid segment encodes a guide RNA targeting the MAP2K1 gene and/or a guide RNA targeting the MAP2K2 gene. In certain embodiments, the guide RNA targeting the MAP2K1 gene targets a nucleic acid sequence having at least 80% identity to any one of SEQ ID NOS: 1-10. In particular embodiments, the guide RNA targeting the MAP2K1 gene targets a nucleic acid sequence having at least 80% identity to SEQ ID NO: 7. In certain embodiments, the guide RNA targeting the MAP2K2 gene targets a nucleic acid sequence having at least 80% identity to any one of SEQ ID NOS: 11-20. In particular embodiments, the guide RNA targeting the MAP2K2 gene targets a nucleic acid sequence having at least 80% identity to SEQ ID NO: 11.

In some embodiments, the first and/or second nucleic acid segment further comprises a promoter. In certain embodiments, the promoter is a Schwann cell-specific promoter. In particular embodiments, the Schwann cell-specific promoter is Myelin protein zero (MPZ, P0) promoter or the peripheral myelin protein 22 (PMP22) P1 promoter.

In a related aspect, the present disclosure provides a vector comprising the polynucleotide comprising (a) a first nucleic acid segment encoding a fusion protein comprising an RNA-guided programmable DNA-binding domain fused to a repressor domain; and (b) a second nucleic acid segment encoding at least one guide RNA targeting a MAP2K gene.

In another aspect, the present disclosure provides a system comprising (a) a first polynucleotide encoding a fusion protein comprising an RNA-guided programmable DNA-binding domain fused to a repressor domain; and (b) a second polynucleotide encoding at least one guide RNA targeting a MAP2K gene.

The present disclosure also provides a cell comprising the polynucleotide or the vector or the system described herein. The present disclosure further provides a composition comprising the polynucleotide or the vector or the system described herein. In some embodiments, the composition comprising the polynucleotide or the vector or the system is formulated as a pharmaceutical composition and further comprising a pharmaceutically acceptable carrier.

In another aspect, the present disclosure provides a method for treating a RASopathy in a subject, comprising administering to the subject a therapeutically effective amount of the pharmaceutical composition described herein. In some embodiments, the RASopathy is selected from the group consisting of Neurofibromatosis type 1 (NF1), Cardio-Facio-Cutaneous (CFC) syndrome, Costello syndrome (CS), Legius syndrome (LS), Capillary malformation-arteriovenous malformation (CM-AVM) syndrome, Noonan syndrome (NS), Noonan syndrome with multiple lentigines (NSML), and Noonan syndrome with loose anagen hair (NSLH). In some embodiments, the RASopathy is Neurofibromatosis type 1 (NF1). In some embodiments, the subject is a human.

In another aspect, the present disclosure provides a method for downregulating a MAP2K gene expression in a subject, comprising administering to the subject a therapeutically effective amount of the pharmaceutical composition described herein. In some embodiments, the subject is suffering a RASopathy disorder. In some embodiments, the RASopathy disorder is Neurofibromatosis type 1 (NF1). In some embodiments, the subject is a human.

In another aspect, the present disclosure provides a guide RNA targeting the MAP2K gene having at least 80% identity to any one of SEQ ID NOS: 1-10. In some embodiments, the guide RNA has at least 80% identity to SEQ ID NO: 7.

In yet another aspect, the present disclosure provides a guide RNA targeting the MAP2K2 gene having at least 80% identity to any one of SEQ ID NOS: 11-20. In some embodiments, the guide RNA has at least 80% identity to SEQ ID NO: 11.

### BRIEF DESCRIPTION OF THE DRAWINGS

FIG. 1 depicts schematic drawings of neurofibromin's function in the RAS/MAPK pathway. Neurofibromin downregulates the Ras signaling pathway by promoting the hydrolysis of the active form of Ras (GTP-bound Ras) to an inactive form of Ras (GDP-bound Ras). With mutated neurofibromin, this inactivation is dysfunctional, leading to increased downstream effect, such as increased cell growth and cell survival, and cancer phenotypes.

FIG. 2 depicts schematic drawings of the epigenetic repression CRISPR-Off/gRNA system. The CRISPR-Off/gRNA system can repress the expression of endogenous genes by fusing dCas9 to a transcriptional repressor domain KRAB and a transcriptional effector domain DNMT3A/3L, forming a CRISPR-Off platform, and the platform is leaded by a gRNA targeting a specific promoter.

FIG. 3 depicts schematic drawings of the steps to develop a CRISPR-based epigenetic repression system. (A) depicts step 1: to identify and develop gene regulatory proteins to reduce MEK1/2 (or MAP2K1 and MAP2K2) expression. To the problem that broadly applicable NF1 treatments are currently not available, a potential solution is to identify CRISPR-based epigenetic regulatory proteins to downregulate MEK1/2. (B) depicts step 2: to produce delivery vehicles to deliver plasmid DNAs (pDNAs) to Schwann cells (SCs). To the problem of no delivery vehicles for large payloads (e.g., >4.7 kb), a potential solution is to use high throughput design, build, test, and learn cycle to produce pDNA delivery vehicles that load and deliver pDNA to Schwann cells in vitro and in vivo. (C) depicts step 3: to test performance of the top 3 candidates in vivo. To the problem that no safe and effective PND-targeted therapy is currently available, a potential solution is to test pDNA payload and top three delivery vehicle candidates for MEK1/2 downloading in mice.

FIG. 4 depicts guide RNA design for MAP2K1 repression. (A) depicts the output of the ChopChop analysis of MAP2K gRNA candidates. (B) depicts the sequences and the distance to transcription start site (TSS) of the 10 selected MAP2K1 gRNAs.

FIG. 5 depicts guide RNA design for MAP2K2 repression. (A) depicts the output of the ChopChop analysis of MAP2K2 gRNA candidates. (B) depicts the sequences and the distance to transcription start site (TSS) of the 10 selected MAP2K2 gRNAs.

FIG. 6 depicts the repression capacities of the selected gRNAs to MAP2K1 and MAP2K2 expression. (A) depicts the relative MAP2K1 repression levels by each of the 10 selected gRNA candidates; (B) depicts the relative MAP2K1 repression levels by each of the selected MAP2K gRNA candidates #6, #7 and #10, or dual gRNAs of MAP2K1 gRNA #6 plus #10, or MAP2K1 gRNA #7 plus #10; (C) depicts the relative MAP2K2 repression levels by each of the 10 selected gRNA candidates; (D) depicts the relative MAP2K2 repression levels by each of the selected MAP2K2 gRNA candidates #1, #7 and #10, or dual gRNAs of MAP2K2 gRNA #1 plus #7, or MAP2K2 gRNA #1 plus #10. MAP2K guide #7 repressed nearly 70% MAP2K expression compared to the control sample without a gRNA, and MAP2K2 guide #1 repressed nearly 90% of MAP2K2 expression, indicating that MAP2K guide #7 and MAP2K2 guide #1 (as arrowed) are the strongest repressors in this CRISPR-Off/gRNA system. A dual gRNA approach yielded either similar repression capabilities, or marginally improved repression compared to a single gRNA.

FIG. 7 depicts schematic showings of a plasmid design for CRISPR-Off/gRNA repression in Schwann cells. The vector carries a polynucleotide encoding a dCas9 protein fused to a transcriptional repressor KRAB domain and an effector DNMT3A/3L domain. The fusion protein is driven by a Schwann cell specific P0 or PMP22 P1 promoter. The polynucleotide also encodes two gRNAs driven by U6 promoter.

FIG. 8 depicts long-term repression of MAP2K1 and MAP2K2 expression using the CRISPR-Off/gRNA system. The CRISPR-Off/MAP2K Guide #7 system repressed about 65% of MAP2K1 expression at day 3 and the repression capacity was plateaued around 60% over the course of 3 weeks. The CRISPR-Off/MAP2K2 Guide #1 system caused about 85% of MAP2K2 expression repression at day 3 and the repression capacity was gradually reduced to nearly 75% over the course of 3 weeks.

### DETAILED DESCRIPTION

### I. Introduction

Recent advances in genome editing provide an opportunity to therapeutically address loss of the tumor suppressor gene neurofibromin 1 (NF1) and prevent neurofibromatosis type 1 (NF1). However, a key challenge that remains to translate an NF1 gene therapy to the clinic is the development of a broadly applicable therapeutic approach for all individuals with NF1 rather than mutation-specific approaches. The present disclosure is focused on creating targeted epigenetic regulatory proteins (ERPs) to be transient therapeutic payloads that cause persistent down-regulation of Mitogen-Activated Protein Kinase Kinases 1 and 2 (MAP2K1/2, or MEK1/2). In particular embodiments, these ERPs can be delivered in the form of plasmid DNA (pDNA) specifically into Schwann cells (SCs) in NF1 patients.

### II. Definitions

Unless specifically indicated otherwise, all technical and scientific terms used herein have the same meaning as commonly understood by those of ordinary skill in the art to which this disclosure belongs. In addition, any method or material similar or equivalent to a method or material described herein can be used in the practice of the present disclosure. For purposes of the present disclosure, the following terms are defined.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The terms “about” and “approximately” shall generally mean an acceptable degree of error for the quantity measured given the nature or precision of the measurements. Typical, exemplary degrees of error are within 20 percent (%), preferably within 10%, and more preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

The terms “subject,” “individual,” and “patient” are used interchangeably herein to refer to a vertebrate, preferably a mammal, more preferably a human. Mammals include, but are not limited to, murines, rats, simians, humans, farm animals, sport animals, and pets. Tissues, cells and their progeny of a biological entity obtained in vivo or cultured in vitro are also encompassed.

As used herein, the term “administering” includes oral administration, topical contact, administration as a suppository, intravenous, intraperitoneal, intramuscular, intralesional, intratumoral, intradermal, intralymphatic, intrathecal, intranasal, or subcutaneous administration to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

The term “treating” refers to an approach for obtaining beneficial or desired results including, but not limited to, a therapeutic benefit and/or a prophylactic benefit. By therapeutic benefit is meant any therapeutically relevant improvement in or effect on one or more diseases, conditions, or symptoms under treatment. Therapeutic benefit can also mean to effect a cure of one or more diseases, conditions, or symptoms under treatment.

The term “effective amount” or “sufficient amount” refers to the amount of a composition that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The specific amount may vary depending on one or more of: the particular agent chosen, the target cell type, the location of the target cell in the subject, the dosing regimen to be followed, whether it is administered in combination with other compounds, timing of administration, and the physical delivery system in which it is carried.

For the purposes herein, an effective amount is determined by such considerations as may be known in the art. The amount must be effective to achieve the desired therapeutic effect in a subject suffering from RASopathy disorders or NF1. The desired therapeutic effect may include, for example, amelioration of undesired symptoms associated with RASopathy disorders or NF1, prevention of the manifestation of such symptoms before they occur, slowing down the progression of symptoms associated with RASopathy disorders or NF1, slowing down or limiting any irreversible damage caused by RASopathy disorders or NF1, lessening the severity of or curing a RASopathy disorders or NF1, or improving the survival rate or providing more rapid recovery from a RASopathy disorders or NF1.

The effective amount depends, inter alia, on the type and severity of the disease to be treated and the treatment regime. The effective amount is typically determined in appropriately designed clinical trials (dose range studies) and the person versed in the art will know how to properly conduct such trials in order to determine the effective amount. As generally known, an effective amount depends on a variety of factors including the distribution profile of a therapeutic agent or composition within the body, the relationship between a variety of pharmacological parameters (e.g., half-life in the body) and undesired side effects, and other factors such as age and gender, etc.

The term “pharmaceutically acceptable carrier” refers to a substance that aids the administration of an active agent to a cell, an organism, or a subject. “Pharmaceutically acceptable carrier” refers to a carrier or excipient that can be included in the compositions of the disclosure and that causes no significant adverse toxicological effect on the subject. Non-limiting examples of pharmaceutically acceptable carriers include water, sodium chloride, normal saline solutions, lactated Ringer's, normal sucrose, normal glucose, binders, fillers, disintegrants, lubricants, coatings, sweeteners, flavors and colors, liposomes, dispersion media, microcapsules, cationic lipid carriers, isotonic and absorption delaying agents, and the like. The carrier may also be substances for providing the formulation with stability, sterility and isotonicity (e.g., antimicrobial preservatives, antioxidants, chelating agents and buffers), for preventing the action of microorganisms (e.g., antimicrobial and antifungal agents, such as parabens, chlorobutanol, sorbic acid and the like) or for providing the formulation with an edible flavor etc. In some instances, the carrier is an agent that facilitates the delivery of CRISPR-based ERPs/gRNAs epigenetic repression system to a target cell or tissue. One of skill in the art will recognize that other pharmaceutical carriers are useful in the present disclosure.

The term “nucleic acid” or “nucleotide” as used herein refers to a polymer containing at least two deoxyribonucleotides or ribonucleotides in either single- or double-stranded form and includes DNA, RNA, and hybrids thereof. DNA may be in the form of, e.g., antisense molecules, plasmid DNA, DNA-DNA duplexes, pre-condensed DNA, PCR products, vectors (P1, PAC, BAC, YAC, artificial chromosomes), expression cassettes, chimeric sequences, chromosomal DNA, or derivatives and combinations of these groups. RNA may be in the form of small interfering RNA (siRNA), Dicer-substrate dsRNA, small hairpin RNA (shRNA), asymmetrical interfering RNA (aiRNA), microRNA (miRNA), mRNA, tRNA, rRNA, tRNA, viral RNA (vRNA), and combinations thereof. Nucleic acids include nucleic acids containing known nucleotide analogs or modified backbone residues or linkages, which are synthetic, naturally occurring, and non-naturally occurring, and which have similar binding properties as the reference nucleic acid. Examples of such analogs include, without limitation, phosphorothioates, phosphoramidates, methyl phosphonates, chiral-methyl phosphonates, 2′-O-methyl ribonucleotides, and peptide-nucleic acids (PNAs). Unless specifically limited, the term encompasses nucleic acids containing known analogues of natural nucleotides that have similar binding properties as the reference nucleic acid. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mol. Cell. Probes, 8:91-98 (1994)). “Nucleotides” contain a sugar deoxyribose (DNA) or ribose (RNA), a base, and a phosphate group. Nucleotides are linked together through the phosphate groups. “Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides.

The term “gene” means the segment of DNA involved in producing a polypeptide chain. The DNA segment may include regions preceding and following the coding region (leader and trailer) involved in the transcription/translation of the gene product and the regulation of the transcription/translation, as well as intervening sequences (introns) between individual coding segments (exons).

The terms “vector” and “expression vector” refer to a nucleic acid construct, generated recombinantly or synthetically, with a series of specified nucleic acid elements that permit transcription of a particular polynucleotide sequence in a host cell. An expression vector may be part of a plasmid, viral genome, or nucleic acid fragment. Typically, an expression vector includes a polynucleotide to be transcribed, operably linked to a promoter. The term “promoter” is used herein to refer to an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. Other elements that may be present in an expression vector include those that enhance transcription (e.g., enhancers) and terminate transcription (e.g., terminators). Gene co-expression may be driven by using a plasmid with multiple, individual expression cassettes. Generally, each promoter creates unique mRNA transcripts for each gene that is expressed. Bicistronic or multicistronic vectors simultaneously express two or more separate proteins from the same mRNA. Bicistronic vectors may contain an Internal Ribosome Entry Site (IRES) to allow for initiation of translation from an internal region of the mRNA. Multicistronic vectors containing one or more self-cleaving 2A peptides are advantageous as they allow gene co-expression from the same cassette. In some instances, multicistronic vectors are preferred when only a portion of the plasmid is packaged for viral delivery, or the relative expression levels between two or more genes is important.

“Recombinant” refers to a genetically modified polynucleotide, polypeptide, cell, tissue, or organism. For example, a recombinant polynucleotide (or a copy or complement of a recombinant polynucleotide) is one that has been manipulated using well known methods. A recombinant expression cassette comprising a promoter operably linked to a second polynucleotide (e.g., a coding sequence) can include a promoter that is heterologous to the second polynucleotide as the result of human manipulation (e.g., by methods described in Sambrook et al., Molecular Cloning—A Laboratory Manual, Cold Spring Harbor Laboratory, Cold Spring Harbor, New York, (1989) or Current Protocols in Molecular Biology Volumes 1-3, John Wiley & Sons, Inc. (1994-1998)). A recombinant expression cassette (or expression vector) typically comprises polynucleotides in combinations that are not found in nature. For instance, human manipulated restriction sites or plasmid vector sequences can flank or separate the promoter from other sequences. A recombinant protein is one that is expressed from a recombinant polynucleotide, and recombinant cells, tissues, and organisms are those that comprise recombinant sequences (polynucleotide and/or polypeptide). A recombinant cell is one that has been modified (e.g., transfected or transformed), with a recombinant nucleotide, expression vector or cassette, or the like.

The term “amino acid” refers to any monomeric unit that can be incorporated into a peptide, polypeptide, or protein. Amino acids include naturally-occurring α-amino acids and their stereoisomers, as well as unnatural (non-naturally occurring) amino acids and their stereoisomers. “Stereoisomers” of a given amino acid refer to isomers having the same molecular formula and intramolecular bonds but different three-dimensional arrangements of bonds and atoms (e.g., an L-amino acid and the corresponding D-amino acid).

Naturally-occurring amino acids are those encoded by the genetic code, as well as those amino acids that are later modified, e.g., hydroxyproline, γ-carboxyglutamate, and O-phosphoserine. Naturally-occurring α-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and their combinations. Stereoisomers of a naturally-occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and their combinations.

Unnatural (non-naturally occurring) amino acids include, without limitation, amino acid analogs, amino acid mimetics, synthetic amino acids, N-substituted glycines, and N-methyl amino acids in either the L- or D-configuration that function in a manner similar to the naturally-occurring amino acids. For example, “amino acid analogs” can be unnatural amino acids that have the same basic chemical structure as naturally-occurring amino acids (i.e., a carbon that is bonded to a hydrogen, a carboxyl group, an amino group) but have modified side-chain groups or modified peptide backbones, e.g., homoserine, norleucine, methionine sulfoxide, methionine methyl sulfonium. “Amino acid mimetics” refer to chemical compounds that have a structure that is different from the general chemical structure of an amino acid, but that functions in a manner similar to a naturally-occurring amino acid. Amino acids may be referred to by either the commonly known three letter symbols or by the one-letter symbols recommended by the IUPAC-IUB Biochemical Nomenclature Commission.

The terms “identity,” “substantial identity,” “similarity,” “substantial similarity,” “homology” and the related terms and expressions used in the context of describing amino acid sequences refer to a sequence that has at least 60% sequence identity to a reference sequence. Examples include at least: 60%, 65%, 70%, 75%, 80%, 85%, 86%, 87%, 88%, 89%, 90%, 91%, 92%, 93%, 94%, 95%, 96%, 97%, 98%, or 99%, sequence identity, as compared to a reference sequence using the programs for comparison of amino acid sequences, such as BLAST using standard parameters. For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default (standard) program parameters can be used, or alternative parameters can be designated. The sequence comparison algorithm then calculates the percent sequence identities for the test sequences relative to the reference sequence, based on the program parameters. A “comparison window” includes reference to a segment of any one of the number of contiguous positions (from 20 to 600, usually about 50 to about 200, more commonly about 100 to about 150), in which a sequence may be compared to a reference sequence of the same number of contiguous positions after the two sequences are optimally aligned. Methods of alignment of sequences for comparison are well-known. Optimal alignment of sequences for comparison may be conducted, for example, by the local homology algorithm of Smith and Waterman, 1981, by the homology alignment algorithm of Needleman and Wunsch, 1970, by the search for similarity method of Pearson and Lipman, 1988, by computerized implementations of these algorithms (for example, BLAST), or by manual alignment and visual inspection.

Algorithms that are suitable for determining percent sequence identity and sequence similarity include BLAST and BLAST 2.0 algorithms, which are described in Altschul et al., 1990, and Altschul et al., 1977, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (Henikoff and Henikoff, 1989). The BLAST algorithm also performs a statistical analysis of the similarity between two sequences (Karlin and Altschul, 1993).

The terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins (i.e., alleles), wherein the amino acid residues are linked by covalent peptide bonds. As used herein, the amino acid sequence of a polypeptide is presented from the N-terminus to the C-terminus. In other words, when describing an amino acid sequence of a polypeptide, the first amino acid at the N-terminus is referred to as the “first amino acid.”

### III. Detailed Description of the Embodiments

The present disclosure relates to the development of an epigenetic approach to treating RASopathies, e.g., neurofibromatosis type 1 (NF1) disorder. Specifically, the present disclosure is focused on creating targeted Epigenetic Regulatory Proteins (ERPs) to be transient therapeutic payloads that cause persistent down-regulation of Mitogen-Activated Protein Kinase Kinases 1 and 2 (MAP2K1/2, or MEK1/2). In particular embodiments, these ERPs can be delivered in the form of plasmid DNA (pDNA) specifically into Schwann cells (SCs) in patients with RASopathies.

In some embodiments, the ERPs include at least one programmable DNA-binding domain and at least one repressor. In some instances, the at least one programmable DNA-binding domain is a dead Cas9 (dCas9) guided by an RNA. In some embodiments, the dCas9 protein is encoded by a nucleic acid sequence having at least 80%, 85%, 90%, 95%, 98%, 99% or 100% identity to SEQ ID NO: 27. In some embodiments, the nucleic acid sequence encoding dCas9 protein comprises the sequence of SEQ ID NO: 27. In some embodiments, the dCas9 protein comprises an amino acid sequence having at least 80%, 85%, 90%, 95%, 98%, 99% or 100% identity to SEQ ID NO: 31. In some embodiments, the dCas9 protein comprises the amino acid sequence of SEQ ID NO: 31. In other instances, the at least one programmable DNA-binding domain can be a zinc-finger nuclease (ZFN) or a modified derivative. In yet other instances, the at least programmable DNA-binding domain can be a transcription activator-like effector (TALE) or a modified derivative.

In some embodiments, the ERPs include at least one repressor domain. The repressor domain includes, but is not limited to, a Krüppel-associated box (KRAB) domain, an mSin3 interaction domain (SID), a four concatenated mSin3 interaction domain (SID4X), a MAX-interacting protein 1 (MXI1), a chromo shadow domain, an EAR-repression domain (SRDX), eukaryotic release factor 1 (ERF1), eukaryotic release factor 3 (ERF3), tetracycline repressor, the lad repressor, Catharanthus roseus G-box binding factors 1 and 2, Drosophila Groucho, Tripartite motif-containing 28 (TRIM28), Nuclear receptor co-repressor 1, Nuclear receptor co-repressor 2, or any combination, fragment, or fusion thereof. In particular embodiments, the repressor comprises a Krüppel-associated box (KRAB) domain. In some embodiments, the KRAB domain is encoded by a nucleic acid sequence having at least 80%, 85%, 90%, 95%, 98%, 99% or 100% identity to SEQ ID NO: 28. In some embodiments, the nucleic acid sequence encoding the KRAB domain comprises the sequence of SEQ ID NO: 28. In some embodiments, the KRAB domain comprises an amino acid sequence having at least 80%, 85%, 90%, 95%, 98%, 99% or 100% identity to SEQ ID NO: 32. In some embodiments, the KRAB domain comprises the amino acid sequence of SEQ ID NO: 32.

In some embodiments, the ERPs further include at least one effector domain. The effector domain includes, but is not limited to, a DNMT3A domain, a DNMT3L domain, a DNMT3A with DNA Methyltransferase 3-Like protein (DNMT3A/3L) domain, a histone-lysine N-methyltransferase SUV39H1, a G9a, an enhancer of zeste homolog 2 (Ezh2), or any combination, fragment, or fusion thereof. In particular embodiments, the effector comprises a DNMT3A domain. In some embodiments, the DNMT3A domain is encoded by a nucleic acid sequence having at least 80%, 85%, 90%, 95%, 98%, 99% or 100% identity to SEQ ID NO: 29. In some embodiments, the nucleic acid sequence encoding the DNMT3A domain comprises the sequence of SEQ ID NO: 29. In some embodiments, the DNMT3A domain comprises an amino acid sequence having at least 80%, 85%, 90%, 95%, 98%, 99% or 100% identity to SEQ ID NO: 33 or 35. In some embodiments, the DNMT3A domain comprises the amino acid sequence of SEQ ID NO: 33 or 35. In some embodiments, the effector comprising a DNMT3A domain further comprises a DNMT3L domain. In some embodiments, the DNMT3L domain is encoded by a nucleic acid sequence having at least 80%, 85%, 90%, 95%, 98%, 99% or 100% identity to SEQ ID NO: 30. In some embodiments, the nucleic acid sequence encoding the DNMT3L domain comprises the sequence of SEQ ID NO: 30. In some embodiments, the DNMT3L domain comprises an amino acid sequence having at least 80%, 85%, 90%, 95%, 98%, 99% or 100% identity to SEQ ID NO: 34 or 36. In some embodiments, the DNMT3L domain comprises the amino acid sequence of SEQ ID NO: 34 or 36.

As disclosed herein, the ERP can be a fusion protein of at least one programmable DNA-binding domain linked to at least one repressor domain, optionally further linked to at least one effector domain. In some embodiments, the ERP fusion protein includes a programmable DNA-binding domain and a repressor. In some embodiments, the ERP fusion protein includes a programmable DNA-binding domain, a repressor, and an effector. The programmable DNA-binding domain, the repressor, and the effector are linked via a flexible linker, thereby their functions are not impacted in the ERP fusion protein. The programmable DNA-binding domain, the repressor, and the effector can be in different orders in the ERP fusion protein. In some embodiments, the programmable DNA-binding domain is guided by an RNA. In some instances, the ERP fusion protein can be in a format of, from N-terminus to C-terminus, an effector domain, an RNA-guided programmable DNA-binding domain, and a repressor domain. In other instances, the ERP fusion protein can be in a format of, from N-terminus to C-terminus, an RNA-guided programmable DNA-binding domain, a repressor domain, and an effector domain. In yet other instances, the ERP fusion protein can be in a format of, from N-terminus to C-terminus, a repressor domain, an effector domain, and an RNA-guided programmable DNA-binding domain. In yet other instances, the ERP fusion protein can be in a format of, from N-terminus to C-terminus, an effector domain, a repressor domain, and an RNA-guided programmable DNA-binding domain.

The ERPs can be also in a form of a multiprotein complex binding together through noncovalent bonds such as electrostatic forces, hydrogen bonding or hydrophobic effect. In some instances, the ERPs can be in a combination of a fusion protein noncovalently binding with one or more proteins.

As disclosed herein, the ERPs can be delivered into a subject in a form of polynucleotide(s). In some instances, the form of polynucleotide is one recombinant polynucleotide including a first nucleic acid segment encoding a fusion protein comprising an RNA-guided programmable DNA-binding domain fused to a repressor domain, optionally further fused to an effector domain, and a second nucleic acid segment encoding at least one guide RNA targeting a MAP2K gene. In other instances, the form of polynucleotide includes two, three, four or more recombinant polynucleotides. In some embodiments, the form of polynucleotide includes two recombinant polynucleotides wherein the first polynucleotide encodes a fusion protein comprising an RNA-guided programmable DNA-binding domain fused to a repressor domain and an effector domain; and the second polynucleotide encodes at least one guide RNA targeting a MAP2K gene. In some embodiments, the first polynucleotide comprises a nucleic acid sequence encoding dCas9 protein comprising a sequence having at least 80%, 85%, 90%, 95%, 98%, 99% or 100% identity to SEQ ID NO: 27. In some embodiments, the first polynucleotide further comprises a nucleic acid sequence encoding a KRAB domain comprising a sequence having at least 80%, 85%, 90%, 95%, 98%, 99% or 100% identity to SEQ ID NO: 28. In some embodiments, the first polynucleotide further comprises a nucleic acid sequence encoding a DNMT3A domain comprising a sequence having at least 80%, 85%, 90%, 95%, 98%, 99% or 100% identity to SEQ ID NO: 29. In some embodiments, the first polynucleotide further comprises a nucleic acid sequence encoding a DNMT3L domain comprising a sequence having at least 80%, 85%, 90%, 95%, 98%, 99% or 100% identity to SEQ ID NO: 30. In some embodiments, the first polynucleotide comprises a nucleic acid sequence comprising SEQ ID NOs: 27-30. The ERPs can be delivered into a subject in a form of fusion protein or a multiprotein complex.

When the ERPs are delivered into a subject in a form of polynucleotide(s), the polynucleotide(s) encoding the ERPs are driven by at least one promoter. In some instances, the promoter is a synthetic promoter, such as CAG. In other instances, the promoter is a cell-specific promoter. In some embodiments, the cell-specific promoter is Schwann cell-specific promoter. In some instances, the Schwann cell-specific promoter is Myelin Protein Zero (MPZ, P0) promoter. In other instances, the Schwann cell-specific promoter is Peripheral Myelin Protein 22 (PMP22) P1 promoter.

The present disclosure is focused on an epigenetic repression therapy specifically downregulating the expression of the MAP2K gene in a subject. The MAP2K gene includes MAP2K1 gene and MAP2K2 gene. The polynucleotide(s) described herein can be used to downregulate both MAP2K and MAP2K2 gene expression by introducing the ERPs together with two gRNAs: the first guide RNA targeting the MAP2K gene and the second guide RNA targeting the MAP2K2 gene. In particular embodiments, the ERPs are delivered into a subject in a form of one recombinant polynucleotide including a first nucleic acid segment encoding a fusion protein comprising an RNA-guided programmable DNA-binding domain fused to a repressor domain, optionally further fused to an effector domain, and a second nucleic acid segment encoding two guide RNAs: one guide RNA targeting the MAP2K1 gene and the other guide RNA targeting the MAP2K2 gene. In other embodiments, the ERPs are delivered into a subject in a form of two recombinant polynucleotides wherein the first polynucleotide encoding a fusion protein comprising an RNA-guided programmable DNA-binding domain fused to a repressor domain and an effector domain; and the second polynucleotide encoding two guide RNAs: one guide RNA targeting the MAP2K1 gene and the other guide RNA targeting the MAP2K2 gene.

In some embodiments, the guide RNA targeting the MAP2K gene targets a nucleic acid sequence comprising at least 80%, 85%, 90%, 95%, 98%, 99% or 100% identity to any one of SEQ ID NOS: 1-10. In particular embodiments, the guide RNA targeting the MAP2K gene targets a nucleic acid sequence comprising at least 80%, 85%, 90%, 95%, 98%, 99% or 100% identity to SEQ ID NO. 7. In some embodiments, the guide RNA targeting the MAP2K2 gene targets a nucleic acid sequence comprising at least 80%, 85%, 90%, 95%, 98%, 99% or 100% identity to any one of SEQ ID NOS: 11-20. In particular embodiments, the guide RNA targeting the MAP2K2 gene targets a nucleic acid sequence comprising at least 80%, 85%, 90%, 95%, 98%, 99% or 100% identity to SEQ ID NO. 11.

In some embodiments, the guide RNA targeting the MAP2K gene comprises at least 80%, 85%, 90%, 95%, 98%, 99% or 100% identity to any one of SEQ ID NOS: 1-10. In particular embodiments, the guide RNA targeting the MAP2K1 gene comprises at least 80%, 85%, 90%, 95%, 98%, 99% or 100% identity to SEQ ID NO. 7. In some embodiments, the guide RNA targeting the MAP2K2 gene comprises at least 80%, 85%, 90%, 95%, 98%, 99% or 100% identity to any one of SEQ ID NOS: 11-20. In particular embodiments, the guide RNA targeting the MAP2K2 gene comprises at least 80%, 85%, 90%, 95%, 98%, 99% or 100% identity to SEQ ID NO. 11.

As disclosed herein, the present disclosure provides a recombinant polynucleotide comprising (a) a first nucleic acid segment encoding a fusion protein comprising an RNA-guided programmable DNA-binding domain fused to a repressor domain; and (b) a second nucleic acid segment encoding at least one guide RNA targeting a MAP2K gene.

In some embodiments, the first nucleic acid segment encodes a fusion protein of dCas9 fused to a KRAB domain. In some embodiments, the first nucleic acid segment comprises a nucleic acid sequence encoding dCas9 comprising a sequence having at least 80%, 85%, 90%, 95%, 98%, 99% or 100% identity to SEQ ID NO: 27 and a nucleic acid sequence encoding a KRAB domain comprising a sequence having at least 80%, 85%, 90%, 95%, 98%, 99% or 100% identity to SEQ ID NO: 28. In particular embodiments, the first nucleic acid segment comprises SEQ ID NOs: 27 and 28. In some embodiments, the first polynucleotide further comprises a nucleic acid sequence encoding a DNMT3A domain comprising a sequence having at least 80%, 85%, 90%, 95%, 98%, 99% or 100% identity to SEQ ID NO: 29. In some embodiments, the first polynucleotide further comprises a nucleic acid sequence encoding a DNMT3L domain comprising a sequence having at least 80%, 85%, 90%, 95%, 98%, 99% or 100% identity to SEQ ID NO: 30. In particular embodiments, the first polynucleotide comprises a nucleic acid sequence comprising SEQ ID NOs: 27-30.

In some instances, the second nucleic acid segment encodes one or more guide RNAs targeting a MAP2K gene. In other instances, the second nucleic acid segment encodes 2, 3, 4, 5, 6, 7, 8, 9, 10, or more guide RNAs targeting a MAP2K gene. In some instances, the second nucleic acid segment encodes one or more guide RNAs targeting the MAP2K1 gene. In some embodiments, the second nucleic acid segment comprises a nucleic acid sequence having at least 80%, 85%, 90%, 95%, 98%, 99% or 100% identity to any one of SEQ ID NOS: 1-10. In some embodiments, the second nucleic acid segment comprises a nucleic acid sequence having at least 80%, 85%, 90%, 95%, 98%, 99% or 100% identity SEQ ID NO: 7. In particular embodiments, the second nucleic acid segment comprises the nucleic acid sequence of SEQ ID NO: 7. In other instances, the second nucleic acid segment encodes one or more guide RNAs targeting the MAP2K2 gene. In some embodiments, the second nucleic acid segment comprises a nucleic acid sequence having at least 80%, 85%, 90%, 95%, 98%, 99% or 100% identity to any one of SEQ ID NOS: 11-20. In some embodiments, the second nucleic acid segment comprises a nucleic acid sequence having at least 80%, 85%, 90%, 95%, 98%, 99% or 100% identity SEQ ID NO: 11. In particular embodiments, the second nucleic acid segment comprises the nucleic acid sequence of SEQ ID NO: 11. In yet other instances, the second nucleic acid segment encodes one or more guide RNAs targeting both the MAP2K1 and MAP2K2 genes. For example, the second nucleic acid segment may comprise a nucleic acid sequence comprising any one of SEQ ID NOS: 1-10 and any one of SEQ ID NOS: 11-20. In particular embodiments, the second nucleic acid segment comprises a nucleic acid sequence comprising both SEQ ID NO: 1 and SEQ ID NO: 11.

The ERPs/gRNAs or the polynucleotide(s) encoding the ERPs/gRNAs described herein can be delivered in one or more expression vectors. A wide variety of viral and non-viral expression vectors may be used, such as plasmids, retroviral vectors, lentiviral vectors, adenoviral vectors, adeno-associated viral vectors, or transposons.

One aspect of the present disclosure provides a cell comprising the ERPs/gRNAs or the polynucleotide(s) encoding the ERPs/gRNAs as described herein. In another aspect, the present disclosure provides a cell comprising an expression vector comprising the ERPs/gRNAs or the polynucleotide(s) encoding the ERPs/gRNAs as described herein.

Another aspect of the present disclosure provides a composition comprising the ERPs/gRNAs or the polynucleotide(s) encoding the ERPs/gRNAs or the expression vector as described herein.

Yet another aspect of the present disclosure provides a pharmaceutical composition comprising the composition as described herein and a pharmaceutically acceptable carrier.

In one aspect, the present disclosure provides a method for treating a RASopathy in a subject comprising administering to the subject a therapeutically effective amount of the pharmaceutical composition as described herein. In some embodiments, the RASopathy is selected from the group consisting of Neurofibromatosis type 1 (NF1), Cardio-Facio-Cutaneous (CFC) syndrome, Costello syndrome (CS), Legius syndrome (LS), Capillary malformation-arteriovenous malformation (CM-AVM) syndrome, Noonan syndrome (NS), Noonan syndrome with multiple lentigines (NSML), and Noonan syndrome with loose anagen hair (NSLH). In particular embodiments, the RASopathy is Neurofibromatosis type 1 (NF1). In some embodiments, the subject is a human.

In another aspect, the present disclosure provides a method for downregulating a MAP2K gene expression in a subject comprising administering to the subject a therapeutically effective amount of the pharmaceutical composition as described herein. In some embodiments, the subject is suffering from a RASopathy disorder. In some embodiments, the RASopathy disorder is Neurofibromatosis type 1 (NF1). In some embodiments, the subject is a human.

### A. RASopathies and Neurofibromatosis Type 1 (NF1)

### RASopathies

RASopathy disorders are one of the largest known groups of malformation syndromes, affecting approximately 1 in 1,000 individuals. These disorders include Neurofibromatosis type 1 (NF1), Cardio-Facio-Cutaneous (CFC) syndrome, Costello syndrome (CS), Legius syndrome (LS), Capillary malformation-arteriovenous malformation (CM-AVM) syndrome, Noonan syndrome (NS), Noonan syndrome with multiple lentigines (NSML), and Noonan syndrome with loose anagen hair (NSLH). These disorders can have overlapping symptoms, but they also have distinctive clinical features. Some of the common symptoms of RASopathy disorders include developmental delays, intellectual disability, facial dysmorphisms, cardiac abnormalities, and an increased risk of certain types of cancer. The severity of symptoms can vary widely, even within the same disorder or among individuals with the same mutation.

RASopathy disorders are caused by various mutations in genes that encode components or regulators of the RAS/MAPK signaling pathway. Neurofibromatosis type 1 (NF1) was the first syndrome of RASopathy identified as being caused by mutation of the neurofibromin 1 (NF1) gene. Noonan syndrome (NS) is caused by activating mutations in PTPN11, SOS1, RAF1, KRAS, NRAS, SHOC2, and CBL. Noonan syndrome with multiple lentigines (NSML) is caused by mutations in PTPN11 and RAF1. Capillary malformation-arteriovenous malformation syndrome (CM-AVM) is caused by haploinsufficiency of RASA1. Costello syndrome (CS) is caused by activating mutations in HRAS. Cardio-facio-cutaneous syndrome (CFC) is caused by alteration of MAPK pathway activation by activating mutations in BRAF and MAP2K1 (MEK1) or MAP2K2 (MEK2). Legius syndrome is caused by inactivating mutations in SPRED1. Mutations in numerous genes in the Ras-MAPK pathway have been shown to cause these disorders, therefore amelioration of any one causative gene might only treat a subset of the affected population. Currently there are no specific treatments for these disorders. The mutations generally result in overactivation of the RAS/MAPK signaling pathway, with the final step consisting of overactive MEKs causing overactivation of ERKs and their many downstream targets. The present disclosure provides a method to downregulate MEK expression epigenetically for a broad treatment to the many causes of RASopathies in patients.

### Neurofibromatosis Type 1 (NF1)

Neurofibromatosis type 1 (NF1) is a complex autosomal dominant disorder caused by germline mutations in the neurofibromin 1 (NF1) gene. Nearly all individuals with NF1 develop pigmentary lesions (café-au-lait macules, skinfold freckling and Lisch nodules) and dermal neurofibromas. Some individuals develop skeletal abnormalities (scoliosis, tibial pseudarthrosis and orbital dysplasia), brain tumors (optic pathway gliomas and glioblastoma), peripheral nerve tumors (spinal neurofibromas, plexiform neurofibromas and malignant peripheral nerve sheath tumors), learning disabilities, attention deficits, and social and behavioral problems, which can negatively affect quality of life. With the identification of NF1 and the generation of accurate preclinical mouse strains that model some of these clinical features, therapies that target the underlying molecular and cellular pathophysiology for neurofibromatosis type 1 are becoming available. No single treatment exists, and current clinical management strategies include early detection of disease phenotypes (risk assessment) and biologically targeted therapies. Similarly, new medical and behavioral interventions are emerging to improve the quality of life of patients. Although considerable progress has been made in understanding this condition, numerous challenges remain.

The average global prevalence of neurofibromatosis type 1 is ~1 case per 3,000 individuals². About 50% of cases of neurofibromatosis type 1 are familial (inherited) and the remainder arise from a de novo NF1 mutation. The life expectancy of individuals with neurofibromatosis type 1 is reduced by ~8-21 years and an excess of deaths occurs in younger individuals (<40 years of age), compared with the general population; the most common cause of early death is malignant neoplasm. Individuals have an increased risk for malignant and non-malignant conditions compared with the general population²⁻⁷.

### NF1 Genetics

The Neurofibromin 1 (NF1) gene, responsible for neurofibromatosis type 1, was identified in 1990, and its function and role in the formation of tumors and the other manifestations of neurofibromatosis type 1 have been under intensive study¹. The gene NF1 encodes neurofibromin, a GTPase-activating protein that negatively regulates RAS/MAPK pathway activity by accelerating the hydrolysis of Ras-bound GTP.

The complexity of molecular testing to identify causative mutations in NF1 is related to the large size of the gene (~60 exons), the relative lack of mutation hotspots and the diversity of the pathogenetic mutations. A multi-step approach is required, with analysis of blood genomic DNA and mRNA, as well as fluorescent in situ hybridization testing for whole NF1 deletions⁸. This strategy identifies >95% of causative mutations, but in people with segmental neurofibromatosis type 1, analysis of affected tissues is necessary as the NF1 mutation is not usually detected in the blood.

To date, >7,000 people with neurofibromatosis type 1 have undergone genetic testing, and >3,000 different germline NF1 mutations have been identified. Although genotype-phenotype correlations are uncommon in neurofibromatosis type 1, three well-established correlations have been identified. Individuals with 1.4 Mb deletions that encompass the entire NF1 gene typically show facial dysmorphism, reduced intellectual abilities and an increased incidence of cancer.⁹ In addition, ~1% of people with neurofibromatosis type 1 have mutations that affect codon 1809 and typically present with café-au-lait macules (CALMs), short stature and pulmonic stenosis, but lack externally visible plexiform or dermal neurofibromas. In addition, another mutation has been associated with an absence of neurofibromas.^{10,11}

The precise mechanisms underlying the development of the clinical manifestations of neurofibromatosis type 1 can vary, such that some manifestations result from haploinsufficiency of NF1, whereas others require biallelic NF1 inactivation or the addition of modifying factors, such as hormones or other genetic alterations. For example, biallelic NF1 inactivation is required for the development of CALMs and neurofibromas, but cooperating genetic alterations, such as TP53 mutation, are required for the formation of malignant peripheral nerve sheath tumors (MPNSTs).

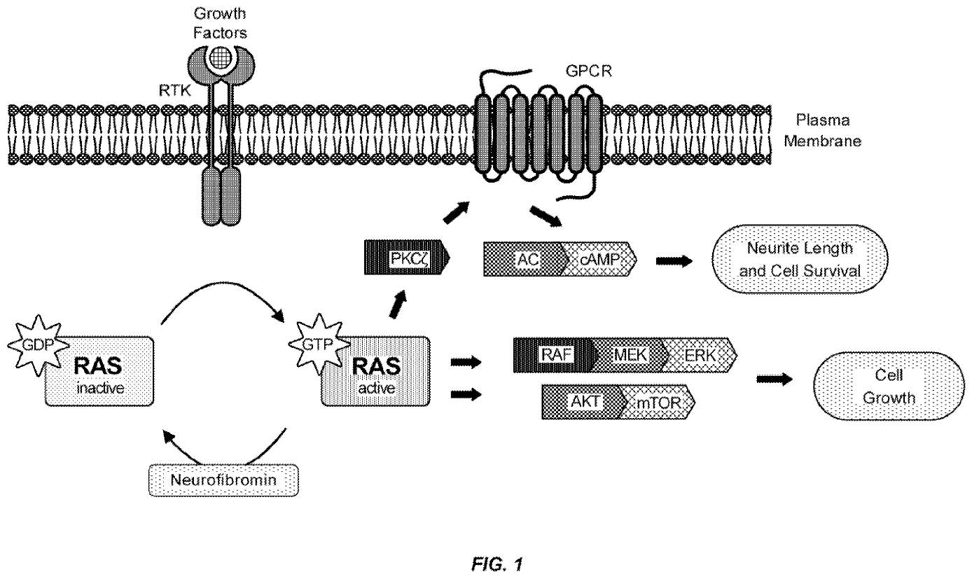

With the discovery of NF1 in 1990, it became possible to envision a future in which treatments might emanate from a deeper understanding of the function of neurofibromin^{12,13}. Neurofibromin is expressed in many cell types, including neurons, glial cells, immune cells, endothelial cells and in cells of the adrenal medulla, but probably functions differently in distinct cell types. Close examination of the predicted amino acid sequence of neurofibromin revealed that a small 300-residue domain of neurofibromin was structurally like a family of proteins that function as negative regulators of the RAS proto-oncogene. These proteins, termed GTPase-activating proteins, inactivate RAS by accelerating the conversion of active GTP-bound RAS to the inactive GDP-bound form. In this manner, loss of neurofibromin expression, as seen in tumors associated with neurofibromatosis type 1, is predicted to lead to increased cell growth and survival through hyperactivation of RAS^{14,15}. RAS then transmits its growth-promoting signal through the AKT-mechanistic target of rapamycin (mTOR) and Rapidly Accelerated Fibrosarcoma (RAF)-Mitogen-activated protein kinase kinase (MAP2K or MEK)-extracellular signal-regulated kinase (ERK) effector pathways^{16,17}, as shown in FIG. 1.

Recent advances in genome editing provide an opportunity to therapeutically address loss of the tumor suppressor gene neurofibromin 1 (NF1) and the treatment for neurofibromatosis type 1 (NF1). However, a key challenge that remains to translate an NF1 gene therapy to the clinic is the development of a broadly applicable therapeutic approach for all individuals with NF1 rather than mutation-specific approaches. The present disclosure provides compositions and methods for preventing or treating a NF1 patient by epigenetically repressing the molecules (e.g., MEKs) in the RAS/MAPK signaling pathway.

### B. MAP2K1 and MAP2K2

The genetic cause of NF1 is mostly due to constitutional heterozygous loss of function mutations of tumor suppressor gene NF. There is somatic inactivation of the wild-type allele in Schwann cells of neurofibromas (according to Knudson 2-hits hypothesis). This explains the hyperactivation of RAS/MAPK pathway in NF1¹⁸. Three major subfamilies of the MAPK pathways have been identified: Stress-activated protein kinases (SAPK)/Jun amino-terminal kinases (JNK), p38 MAPK, and MEK/ERK. Both MEK and ERK are critical components of the RAS-regulated RAF/MEK/ERK pathway, which is often activated in different types of cancers.

MEK, also known as Mitogen-Activated Protein Kinase Kinase (MAP2K), is an upstream regulator of the ERK pathway. MEK(MAP2K) contains two consensus kinase motifs, MAP2K1 and MAP2K2. MAP2K1 and MAP2K2, encoded by the MAP2K1 and MAP2K2 genes, are involved in the phosphorylation of serine/threonine and tyrosine residues of the downstream kinases ERKs, which has multiple downstream effectors involved in a number of cellular functions including transcription, cell cycle progression and cell motility. Once activated, ERKs, along with Rapidly Accelerated Fibrosarcoma (RAF) and MAP2K, migrate to the nucleus where they activate cyclin D1 and down regulate p27 thus driving cell proliferation. Activation may also lead to inhibition of apoptosis or activation of anti-apoptotic proteins¹⁹.

The present disclosure provides compositions and methods to create targeted epigenetic regulatory proteins (ERPs) to be transient therapeutic payloads that cause persistent down-regulation of mitogen-activated protein kinase kinases 1 and 2 (MAP2K1/2 or MEK1/2) and to deliver plasmid DNA (pDNA) encoding these ERPs efficiently into Schwann cells (SCs) in a patient.

### C. Epigenetic Repression

Epigenetic repression refers to a process by which genes are silenced or turned off without any change to the underlying DNA sequence. This repression is achieved through various epigenetic modifications, such as DNA methylation, histone modification, and non-coding RNA regulation.

DNA methylation involves the addition of a methyl group to the cytosine base of DNA, which can alter gene expression patterns by preventing the transcriptional machinery from accessing the DNA sequence. Histone modification involves the addition or removal of chemical groups to histone proteins, which can change how tightly the DNA is wrapped around the histone proteins and, consequently, affect the accessibility of the DNA to transcription factors. Non-coding RNA regulation involves small RNA molecules that can bind to messenger RNA (mRNA) and prevent translation into protein.

CRISPR can be also used for epigenetic modification. One approach to using CRISPR for epigenetic repression involves using the dCas9 protein, which is a modified version of Cas9 that lacks the ability to cut DNA. The dCas9 protein is fused with an epigenetic modifier, such as a methyltransferase or histone deacetylase, and guided by an RNA targeting to specific regions of the genome to selectively repress gene expression without altering the underlying DNA sequence.

### 1. CRISPR Repression

As disclosed herein, a CRISPR repression system termed CRISPR-Off²¹ was developed to repress the expression of MAP2K1 and MAP2K2 genes. CRISPR-Off is a programmable epigenetic memory writer consisting of a single dead Cas9 (dCas9) fusion protein that establishes DNA methylation and repressive histone modifications. Transient CRISPR-Off expression initiates highly specific DNA methylation and gene repression that has been shown to be maintained through cell division at some genetic loci²².

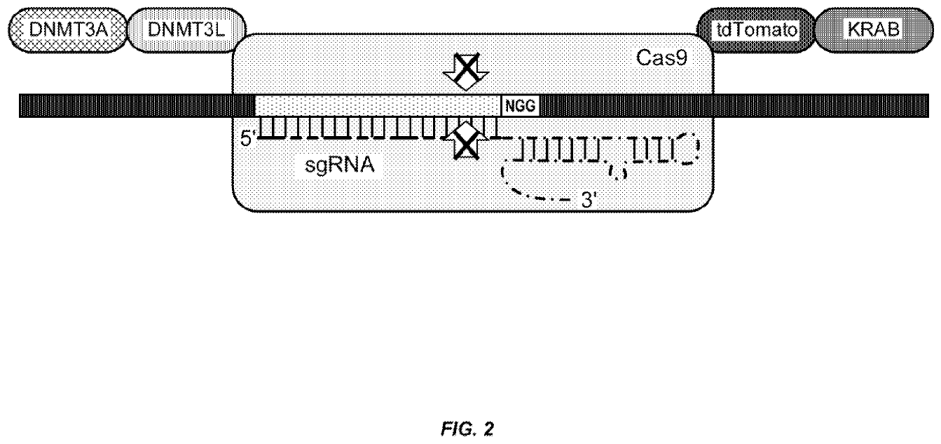

CRISPR repression can be achieved by fusing a Krüppel-associated box (KRAB) domain to a dead Cas9 (dCas9) protein, which is then targeted to a specific genomic locus using guide RNAs (FIG. 2). The KRAB domain interacts with endogenous co-repressor proteins, such as KAP1, which recruit chromatin-modifying enzymes, including DNA methyltransferase 3A (DNMT3A) and lysine-specific histone demethylase 1A (LSD1), to the targeted genomic locus. DNMT3A then catalyzes the addition of a methyl group to cytosine residues in the DNA, leading to transcriptional repression by preventing the binding of transcription factors and other regulatory proteins.

CRISPR repression can be further enhanced by fusing to the KRAB-dCas9 system an effector domain, such as a DNMT3A domain. The DNMT3A effector domain can further include a DNMT3L domain (FIG. 2). The DNMT3A/3L domain is a catalytically active DNA methyltransferase domain that can also methylate cytosine residues in the targeted genomic locus. Fusing the DNMT3A/3L domain to KRAB-dCas9 can enhance transcriptional repression, leading to more efficient gene silencing. Overall, the KRAB-dCas9 system with DNMT3A/3L effector domain is a powerful tool for achieving targeted gene repression and has potential applications in gene therapy and genetic research^{23,24}.

2. Cas9 and dCas9

In some embodiments, the Epigenetic Regulatory Proteins (ERPs) used in methods and compositions of the disclosure includes a nuclease-deficient Cas protein. A Cas protein refers to a clustered regularly interspaced short palindromic repeats (CRISPR)-associated protein. Wild-type Cas protein has two functional domains, e.g., RuvC and HNH, that cut different DNA strands. A Cas protein can induce double-stranded breaks in genomic DNA (target nucleic acid) when both functional domains are active. The Cas protein can comprise one or more catalytic domains of a Cas protein derived from bacteria belonging to the group consisting of Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, and Campylobacter. In some embodiments, the Cas protein can be a fusion protein, e.g., the two catalytic domains are derived from different bacteria species.

Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, homologs thereof, variants thereof, mutants thereof, and derivatives thereof. There are three main types of Cas proteins (type I, type II, and type III), and 10 subtypes including 5 type I, 3 type II, and 2 type III proteins (see, e.g., Hochstrasser and Doudna, Trends Biochem Sci, 2015:40(1):58-66). Type II Cas proteins include Cas1, Cas2, Csn2, Cas9, and Cfp1. These Cas proteins are known to those skilled in the art. For example, the amino acid sequence of the Streptococcus pyogenes wild-type Cas9 polypeptide is set forth, e.g., in NBCI Ref Seq. No. NP_269215, and the amino acid sequence of Streptococcus thermophilus wild-type Cas9 polypeptide is set forth, e.g., in NBCI Ref. Seq. No. WP_011681470.

Cas proteins, e.g., Cas9 protein, can be derived from a variety of bacterial species including, but not limited to, Veillonella atypical, Fusobacterium nucleatum, Filifactor alocis, Solobacterium moorei, Coprococcus catus, Treponema denticola, Peptoniphilus duerdenii, Catenibacterium mitsuokai, Streptococcus mutans, Listeria innocua, Staphylococcus pseudintermedius, Acidaminococcus intestine, Olsenella uli, Oenococcus kitaharae, Bifidobacterium bifidum, Lactobacillus rhamnosus, Lactobacillus gasseri, Finegoldia magna, Mycoplasma mobile, Mycoplasma gallisepticum, Mycoplasma ovipneumoniae, Mycoplasma canis, Mycoplasma synoviae, Eubacterium rectale, Streptococcus thermophilus, Eubacterium dolichum, Lactobacillus coryniformis subsp. Torquens, Ilyobacter polytropus, Ruminococcus albus, Akkermansia muciniphila, Acidothermus cellulolyticus, Bifidobacterium longum, Bifidobacterium dentium, Corynebacterium diphtheria, Elusimicrobium minutum, Nitratifractor salsuginis, Sphaerochaeta globus, Fibrobacter succinogenes subsp. Succinogenes, Bacteroides fragilis, Capnocytophaga ochracea, Rhodopseudomonas palustris, Prevotella micans, Prevotella ruminicola, Flavobacterium columnare, Aminomonas paucivorans, Rhodospirillum rubrum, Candidatus Puniceispirillum marinum, Verminephrobacter eiseniae, Ralstonia syzygii, Dinoroseobacter shibae, Azospirillum, Nitrobacter hamburgensis, Bradyrhizobium, Wolinella succinogenes, Campylobacter jejuni subsp. Jejuni, Helicobacter mustelae, Bacillus cereus, Acidovorax ebreus, Clostridium perfringens, Parvibaculum lavamentivorans, Roseburia intestinalis, Neisseria meningitidis, Pasteurella multocida subsp. Multocida, Sutterella wadsworthensis, proteobacterium, Legionella pneumophila, Parasutterella excrementihominis, Wolinella succinogenes, and Francisella novicida.

In the embodiments described herein, the Cas protein is a nuclease-inactive variant. For example, useful variants of the Cas9 nuclease can include a single inactive catalytic domain, such as a RuvC- or HNH-enzyme or a nickase. A Cas9 nickase has only one active functional domain and can cut only one strand of the target nucleic acid, thereby creating a single strand break or nick. In some embodiments, the Cas9 nuclease can be a mutant Cas9 nuclease having one or more amino acid mutations. For example, the mutant Cas9 having at least a D10A mutation is a Cas9 nickase. In other embodiments, the mutant Cas9 nuclease having at least a H840A mutation is a Cas9 nickase. Other examples of mutations present in a Cas9 nickase include, without limitation, N854A and N863A. A double-strand break can be introduced using a Cas9 nickase if at least two DNA-targeting RNAs that target opposite DNA strands are used. A double-nicked induced double-strand break can be repaired by NHEJ or HDR (Ran et al., 2013, Cell, 154:1380-1389). Non-limiting examples of Cas9 nucleases or nickases are described in, for example, U.S. Pat. Nos. 8,895,308; 8,889,418; and 8,865,406 and U.S. Application Publication Nos. 2014/0356959, 2014/0273226 and 2014/0186919. The Cas9 nuclease or nickase can be codon-optimized for the target cell or target organism.

In some embodiments, a catalytically-inactive Cas protein variant can be a Cas9 polypeptide that contains two silencing mutations of the RuvC1 and HNH nuclease domains (D10A and H840A), which is referred to as dCas9 (Jinek et al., Science, 2012, 337:816-821; Qi et al., Cell, 152(5):1173-1183). In one embodiment, the dCas9 polypeptide from Streptococcus pyogenes comprises at least one mutation at position D10, G12, G17, E762, H840, N854, N863, H982, H983, A984, D986, A987 or any combination thereof. Descriptions of such dCas9 polypeptides and variants thereof are provided in, for example, International Patent Publication No. WO 2013/176772. The dCas9 enzyme can contain a mutation at D10, E762, H983, or D986, as well as a mutation at H840 or N863. In some instances, the dCas9 enzyme can contain a D10A or D10N mutation. Also, the dCas9 enzyme can contain a H840A, H840Y, or H840N. In some embodiments, the dCas9 enzyme can contain D10A and H840A; D10A and H840Y; D10A and H840N; DION and H840A; DION and H840Y; or DION and H840N substitutions. The substitutions can be conservative or non-conservative substitutions to render the Cas9 polypeptide catalytically inactive and able to bind to target nucleic acid. In some embodiments, the dCas9 protein is encoded by a nucleic acid sequence having at least 80%, 85%, 90%, 95%, 98%, 99% or 100% identity to SEQ ID NO: 27. In some embodiments, the nucleic acid sequence encoding dCas9 protein comprises the sequence of SEQ ID NO: 27. In some embodiments, the dCas9 protein comprises an amino acid sequence having at least 80%, 85%, 90%, 95%, 98%, 99% or 100% identity to SEQ ID NO: 31. In some embodiments, the dCas9 protein comprises the amino acid sequence of SEQ ID NO: 31.

In the methods and compositions described herein, a catalytically-inactive Cas protein (e.g., dCas9) can be linked to a transcriptional repressor domain (e.g., KRAB) to target a promoter or enhancer sequence of the gene of interest in order to downregulate the expression of the gene.

As described herein, the programmable DNA-binding domain refers to any polypeptide binding to a DNA. In some embodiments, the programmable DNA-binding domain can be a catalytically-inactive CRISPR nuclease. In other instances, the programmable DNA-binding domain can be a zinc-finger nuclease (ZFN) or a modified derivative. In yet other instances, the programmable DNA-binding domain can be a transcription activator-like effector (TALE) or a modified derivative. In some embodiments, the programmable DNA-binding domain is dCas9 guided by an RNA. The CRISPR repression (CRISPRr) system utilizes a catalytically-inactive CRISPR nuclease (e.g. dCas9) linked to one or more transcriptional repressors (e.g. KRAB) to downregulate expression of the genes of interest. The CRISPRr system can further include an effector domain. For example, the dCas9-KRAB protein can be further fused to a DNMT3A and DNMT3L domain that catalyzes de novo methylation to cytosine residues in specific DNA regions, leading to enhancing transcriptional repression by preventing the binding of transcription factors and other regulatory proteins into the DNA. The CRISPRr system further includes one or more guide RNAs targeting the gene of interest whose expression is to be downregulated (e.g., the MAP2K1 or MAP2K2 gene).

3. gRNAs

A guide RNA (gRNA) is aversion of the naturally occurring two-piece RNA (crRNA and tracrRNA) engineered into a two-piece gRNA or a single, continuous sequence. A gRNA can contain a guide sequence (e.g., the crRNA equivalent portion of the gRNA) that targets the Cas protein to the target nucleic acid and a scaffold sequence that interacts with the Cas protein (e.g., the tracrRNAs equivalent portion of the gRNA). A gRNA can be selected using a software. As a non-limiting example, considerations for selecting a gRNA can include, e.g., the PAM sequence for the Cas protein to be used, and strategies for minimizing off-target modifications. Tools, such as ChopChop, NUPACK® or the CRISPR Design Tool, can provide sequences for preparing the gRNA, for assessing target modification efficiency, and/or assessing cleavage at off-target sites.

### Guide Sequence

The guide sequence in the gRNA may be complementary to a specific sequence within a target nucleic acid (e.g., the MAP2K or MAP2K2 gene). The 3′ end of the target nucleic acid sequence can be followed by a PAM sequence. Approximately 20 nucleotides upstream of the PAM sequence is the target nucleic acid. In general, a Cas9 protein or a variant thereof cleaves about three nucleotides upstream of the PAM sequence. The guide sequence in the gRNA can be complementary to either strand of the target nucleic acid.

In some embodiments, the guide sequence of a gRNA comprises about 100 nucleic acids at the 5′ end of the gRNA that can direct the Cas protein to the target nucleic acid site using RNA-DNA complementarity base pairing. In some embodiments, the guide sequence comprises about 20 nucleic acids at the 5′ end of the gRNA that can direct the Cas protein to the target nucleic acid site using RNA-DNA complementarity base pairing. In other embodiments, the guide sequence comprises less than 20, e.g., 19, 18, 17, 16, 15 or less, nucleic acids that are complementary to the target nucleic acid site. In some instances, the guide sequence in the gRNA contains at least one nucleic acid mismatch in the complementarity region of the target nucleic acid site. In some instances, the guide sequence contains about 1 to about 10 nucleic acid mismatches in the complementarity region of the target nucleic acid site.

### Scaffold Sequence

The scaffold sequence in the gRNA can serve as a protein-binding sequence that interacts with the Cas protein or a variant thereof. In some embodiments, the scaffold sequence in the gRNA can comprise two complementary stretches of nucleotides that hybridize to one another to form a double-stranded RNA duplex (dsRNA duplex). The scaffold sequence may have structures such as lower stem, bulge, upper stem, nexus, and/or hairpin. In some embodiments, the scaffold sequence in the gRNA can be between about 90 nucleic acids to about 120 nucleic acids.

As described herein, the CRISPRr system is guided by one or more gRNAs targeting the MAP2K1 or MAP2K2 gene. In some embodiments, the guide RNA targeting the MAP2K1 gene targets a nucleic acid sequence comprising at least 80%, 85%, 90%, 95%, 98%, 99% or 100% identity to any one of SEQ ID NOS: 1-10. In particular embodiments, the guide RNA targeting the MAP2K gene targets a nucleic acid sequence comprising at least 80%, 85%, 90%, 95%, 98%, 99% or 100% identity to SEQ ID NO. 7. In some embodiments, the guide RNA targeting the MAP2K2 gene targets a nucleic acid sequence comprising at least 80%, 85%, 90%, 95%, 98%, 99% or 100% identity to any one of SEQ ID NOS: 11-20. In particular embodiments, the guide RNA targeting the MAP2K2 gene targets a nucleic acid sequence comprising at least 80%, 85%, 90%, 95%, 98%, 99% or 100% identity to SEQ ID NO. 11.

In some embodiments, the guide RNA targeting the MAP2K1 gene comprises at least 80%, 85%, 90%, 95%, 98%, 99% or 100% identity to any one of SEQ ID NOS: 1-10. In particular embodiments, the guide RNA targeting the MAP2K1 gene comprises at least 80%, 85%, 90%, 95%, 98%, 99% or 100% identity to SEQ ID NO. 7. In some embodiments, the guide RNA targeting the MAP2K2 gene comprises at least 80%, 85%, 90%, 95%, 98%, 99% or 100% identity to any one of SEQ ID NOS: 11-20. In particular embodiments, the guide RNA targeting the MAP2K2 gene comprises at least 80%, 85%, 90%, 95%, 98%, 99% or 100% identity to SEQ ID NO. 11.

### 4. Repressors

The terms “repressor” and “repressor domain” are used interchangeably herein to refer to a DNA- or RNA-binding polypeptide that forms a stable 3D structure and inhibits the expression of one or more genes by binding to the operator or associated silencers. A DNA-binding repressor blocks the attachment of RNA polymerase to the promoter, thus preventing transcription of the genes into messenger RNA. An RNA-binding repressor binds to the mRNA and prevents translation of the mRNA into protein. This blocking or reducing of expression is called repression. As disclosed herein, an ERP fusion protein may comprise a repressor domain (e.g., KRAB domain).

The repressor may be any known repressor of gene expression, for example, a repressor chosen from Krüppel associated box (KRAB) domain, mSin3 interaction domain (SID), a four concatenated mSin3 interaction domain (SID4X), MAX-interacting protein 1 (MXI1), a chromo shadow domain, an EAR-repression domain (SRDX), eukaryotic release factor 1 (ERF1), eukaryotic release factor 3 (ERF3), tetracycline repressor, the lad repressor, Catharanthus roseus G-box binding factors 1 and 2, Drosophila Groucho, Tripartite motif-containing 28 (TRIM28), Nuclear receptor co-repressor 1, Nuclear receptor co-repressor 2, any combination, fragment, or fusion thereof.

### Kruppel Associated Box (KRAB)

The Krüppel associated box (KRAB) domain is a category of transcriptional repression domains present in approximately 400 human zinc finger protein-based transcription factors (KRAB-ZFPs). The KRAB domain typically consists of about 75 amino acid residues, while the minimal repression module is approximately 45 amino acid residues. The KRAB domain contains two subdomains: A and B. Subdomain A is highly conserved and forms an alpha helix, while subdomain B is more variable and forms a beta strand. The KRAB domain interacts with a co-repressor protein, KRAB-associated protein 1 (KAP1), and heterochromatin protein 1 (HP1), as well as other chromatin modulating proteins, leading to transcriptional repression through heterochromatin formation. The KRAB domain is highly conserved. Substitutions for the conserved residues abolish repression.

The KRAB domains are involved in a wide range of biological processes, including embryonic development, cell differentiation, and immune response. Dysregulation of KRAB domains has been linked to several human diseases, including cancer and developmental disorders.

Over 10 independently encoded KRAB domains have been shown to be effective repressors of transcription, demonstrating this activity to be a common property of the domain. KRAB domains can be fused with dCas9 to form stronger repressors. In some embodiments, the KRAB domain is encoded by a nucleic acid sequence having at least 80%, 85%, 90%, 95%, 98%, 99% or 100% identity to SEQ ID NO: 28. In some embodiments, the nucleic acid sequence encoding the KRAB domain comprises the sequence of SEQ ID NO: 28. In some embodiments, the KRAB domain comprises an amino acid sequence having at least 80%, 85%, 90%, 95%, 98%, 99% or 100% identity to SEQ ID NO: 32. In some embodiments, the KRAB domain comprises the amino acid sequence of SEQ ID NO: 32.

As disclosed herein, the CRISPR repression system includes a fusion protein that a Krüppel-associated box (KRAB) domain is fused to a dead Cas9 (dCas9) protein, which is then targeted to a specific genomic locus using guide RNAs. The KRAB domain interacts with endogenous co-repressor proteins, such as KAP1, which recruit chromatin-modifying enzymes, including DNA methyltransferase 3A (DNMT3A) and lysine-specific histone demethylase 1A (LSD1), to the targeted genomic locus. DNMT3A then catalyzes the addition of a methyl group to cytosine residues in the DNA, leading to transcriptional repression by preventing the binding of transcription factors and other regulatory proteins.

### 5. Effectors

The terms “effector” and “effector domain” are used interchangeably herein to refer to a polypeptide assisting or contributing repression of one or more genes. As disclosed herein, an effector may be a DNA methyltransferase. In some embodiments, the effector comprises a DNA methyltransferase 3A (DNMT3A) domain, a DNMT3A with DNA Methyltransferase 3-Like protein (DNMT3A/3L) domain, a histone-lysine N-methyltransferase SUV39H1, a G9a, an enhancer of zeste homolog 2 (Ezh2), or any combination, fragment, or fusion thereof. In some embodiments, the effector comprises a DNA methyltransferase 3A (DNMT3A). In particular embodiments, the effector comprises a DNMT3A/3L domain.

### DNMT3A and DNMT3L

DNA Methyltransferase 3A (DNMT3A) and its regulatory factor, DNA Methyltransferase 3-Like protein (DNMT3L) are two important proteins that play a role in DNA methylation, a process of adding a methyl group to the cytosine nucleotide in DNA, which can affect gene expression.

DNMT3A is a member of the DNA methyltransferase family, which consists of the protagonists DNMT1, DNMT3A and DNMT3B that catalyze the transfer of a methyl group to DNA. DNMT3A specifically catalyzes de novo methylation, meaning it adds methyl groups to previously unmethylated DNA regions, which enables key epigenetic modifications essential for processes such as cellular differentiation and embryonic development, transcriptional regulation, heterochromatin formation, X-inactivation, imprinting and genome stability. DNMT3A is important in the regulation of gene expression during development and in maintaining the integrity of genomic imprinting. Mutations in the DNMT3A gene have been associated with various diseases, including acute myeloid leukemia.

DNMT3A is a 130 kDa protein encoded by 23 exons found on chromosome 2p23 in humans. There exists a 98% homology between human and murine homologues. DNMT3A consists of three major protein domains: the Pro-Trp-Trp-Pro (PWWP) domain, the ATRX-DNMT3-DNMT3L (ADD) domain and the catalytic methyltransferase domain. The ADD domain serves as an inhibitor of the methyltransferase domain until DNMT3A binds to the unmodified lysine 4 of histone 3 (H3K4me0) for its de novo methylating activity. This protein thus seems to have an inbuilt control mechanism targeting histones only for methylation. Finally, the methyltransferase domain is highly conserved, even among prokaryotes.

DNMT3L is a regulatory protein that interacts with DNMT3A and enhances its activity. DNMT3L is necessary for the establishment of maternal genomic imprinting during early development. While DNMT3A can catalyze de novo methylation on its own, it is more efficient with the presence of DNMT3L. DNMT3L does not have catalytic activity, but it can target DNMT3A to specific DNA regions. DNMT3L also mediates transcriptional repression through interaction with histone deacetylase 1 (HDAC1).

As disclosed herein, in some instances, the effector domain is a DNMT3A. In some embodiments, the DNMT3A is encoded by a nucleic acid sequence having at least 80%, 85%, 90%, 95%, 98%, 99% or 100% identity to SEQ ID NO: 29. In some embodiments, the nucleic acid sequence encoding the DNMT3A comprises the sequence of SEQ ID NO: 29. In some embodiments, the DNMT3A comprises an amino acid sequence having at least 80%, 85%, 90%, 95%, 98%, 99% or 100% identity to SEQ ID NO: 33 or 35. In some embodiments, the DNMT3A protein comprises the amino acid sequence of SEQ ID NO: 33 or 35. In other instances, the effector domain is a DNMT3L. In some embodiments, the DNMT3L is encoded by a nucleic acid sequence having at least 80%, 85%, 90%, 95%, 98%, 99% or 100% identity to SEQ ID NO: 30. In some embodiments, the nucleic acid sequence encoding the DNMT3L comprises the sequence of SEQ ID NO: 30. In some embodiments, the DNMT3L comprises an amino acid sequence having at least 80%, 85%, 90%, 95%, 98%, 99% or 100% identity to SEQ ID NO: 34 or 36. In some embodiments, the DNMT3L protein comprises the amino acid sequence of SEQ ID NO: 34 or 36. In yet other instances, the effector domain comprises both DNMT3A and DNMT3L.

As disclosed herein, the CRISPR repression system can be further enhanced by fusing an effector domain to the KRAB-dCas9 protein. The effector domain can be a DNMT3A domain, or a DNMT3A domain fused with a DNMT3L domain (DNMT3A/3L domain) (FIG. 2). Fusing the DNMT3A/3L domain to KRAB-dCas9 can enhance transcriptional repression, leading to more efficient gene silencing. The KRAB-dCas9 system with DNMT3A/3L effector domain can be a powerful tool for achieving targeted gene repression and has potential applications in gene therapy and genetic research.

### D. Schwann Cells (SCs) and SC-Specific Promoters

Schwann cells (SCs) are the major glial cell type in the peripheral nervous system. They play essential roles in the development, maintenance, function, and regeneration of peripheral nerves. The two types of Schwann cells are myelinating and non-myelinating. Myelinating Schwann cells wrap around axons of motor and sensory neurons to form the myelin sheath. The Schwann cell promoter is present in the downstream region of the human dystrophin gene that gives shortened transcript that are synthesized in a tissue-specific manner.

Several Schwann cell-specific promoters have been identified and characterized, including the myelin protein zero (PMZ or P0) promoter, the peripheral myelin protein 22 (PMP22) P1 promoter, and the myelin basic protein (MBP) promoter.

The myelin protein zero (PMZ or P0), driven by the P0 promoter, is expressed at significant levels specifically in Schwann cells. This transmembrane glycoprotein of the immunoglobulin superfamily is detected in neural crest cells committed to the glial lineage and continues to be present throughout development of the Schwann cell lineage at low levels. Upon myelination, P0 expression is massively upregulated. As a consequence, P0 makes up more than 50% of the total myelin protein in mature Schwann cells, where it is directly involved in myelin compaction. Therefore, the P0 promoter is an attractive target for the analysis of cell-specific transcriptional regulation.

The peripheral myelin protein 22 (PMP22) is expressed from two promoters P1 and P2. Both the P1 and P2 promoters are upregulated during myelination, but only P1 promoter is Schwann cell specific. The P1 promoter driving exon 1A expression is largely Schwann cell-specific and accounts for half of Schwann cell Pmp22 transcripts in humans and ~75% of transcripts in rodents. The P2 promoter driving exon 1B-containing transcripts is the primary promoter used in other tissues, such as brain, heart, lung, and gut. Transfection studies of Pmp22 expression have shown that the P1 promoter can drive expression of a reporter in Schwann cells.

### E. Pharmaceutical Formulations

The compositions (e.g., expression cassettes, or cells having upregulated or overexpressed gene products) described herein can be formulated together with a pharmaceutically acceptable carrier. The compositions can additionally contain other therapeutic agents that are suitable for treating or preventing a given disorder. Pharmaceutically carriers can enhance or stabilize the composition, or to facilitate preparation of the composition. Pharmaceutically acceptable carriers include solvents, dispersion media, coatings, antibacterial and antifungal agents, isotonic and absorption delaying agents, and the like that are physiologically compatible.

A pharmaceutical composition as described herein can be administered by a variety of methods known in the art. The route and/or mode of administration vary depending upon the desired results. In some embodiments, the composition is sterile and fluid. Proper fluidity can be maintained, for example, by use of coating such as lecithin, by maintenance of required particle size in the case of dispersion and by use of surfactants. In many cases, it is preferable to include isotonic agents, for example, sugars, polyalcohol such as mannitol or sorbitol, and sodium chloride in the composition. Long-term absorption of the injectable compositions can be brought about by including in the composition an agent which delays absorption, for example, aluminum monostearate or gelatin.

Pharmaceutical compositions described herein can be prepared in accordance with methods well known and routinely practiced in the art. Pharmaceutically acceptable carriers are determined in part by the particular composition being administered, as well as by the particular method used to administer the composition. Accordingly, there is a wide variety of suitable formulations of pharmaceutical compositions. Applicable methods for formulating the compositions and determining appropriate dosing and scheduling can be found, for example, in Remington: The Science and Practice of Pharmacy, 21^{st} Ed., University of the Sciences in Philadelphia, Eds., Lippincott Williams & Wilkins (2005); and in Martindale: The Complete Drug Reference, Sweetman, 2005, London: Pharmaceutical Press, and in Martindale, Martindale: The Extra Pharmacopoeia, 31st Edition., 1996, Amer Pharmaceutical Assn, and Sustained and Controlled Release Drug Delivery Systems, J. R. Robinson, ed., Marcel Dekker, Inc., New York, 1978, each of which are hereby incorporated herein by reference. Pharmaceutical compositions are preferably manufactured under GMP conditions. Typically, a therapeutically effective dose or efficacious dose is employed in the pharmaceutical compositions described herein. The compositions can be formulated into pharmaceutically acceptable dosage forms. Dosage regimens are adjusted to provide the desired response (e.g., a therapeutic response). In determining a therapeutically or prophylactically effective dose, a low dose can be administered and then incrementally increased until a desired response is achieved with minimal or no undesired side effects. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

Actual dosage levels of the active ingredients in the pharmaceutical compositions can be varied so as to obtain an amount of the active ingredient which is effective to achieve the desired therapeutic response for a particular patient, composition, and mode of administration, without being toxic to the patient. The selected dosage level depends upon a variety of pharmacokinetic factors including the activity of the particular compositions employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors.

### F. Methods of Treatment

The present disclosure provides a method for treating a RASopathy in a subject. In some embodiments, the method comprises administering to the subject a therapeutically effective amount of a pharmaceutical composition of the present disclosure (e.g., a pharmaceutical composition comprising recombinant polynucleotide(s) encoding the ERPS and at least one guide RNA targeting a MAP2K gene of the present disclosure) described herein.

In some embodiments, the RASopathy is selected from the group consisting of Neurofibromatosis type 1 (NF1), Cardio-Facio-Cutaneous (CFC) syndrome, Costello syndrome (CS), Legius syndrome (LS), Capillary malformation-arteriovenous malformation (CM-AVM) syndrome, Noonan syndrome (NS), Noonan syndrome with multiple lentigines (NSML), and Noonan syndrome with loose anagen hair (NSLH). In some embodiments, the RASopathy is Neurofibromatosis type 1 (NF1). In some embodiments, the subject is a human.

In another aspect, the present disclosure provides a method for downregulating a MAP2K gene expression in a subject comprising administering to the subject a therapeutically effective amount of the pharmaceutical composition as described herein. In some embodiments, the subject is suffering a RASopathy disorder. In some embodiments, the RASopathy disorder is Neurofibromatosis type 1 (NF1). In some embodiments, the subject is a human.

### IV. Examples

The present disclosure will be described in greater detail by way of specific examples. The following examples are offered for illustrative purposes only, and are not intended to limit the invention in any manner. Those of skill in the art will readily recognize a variety of noncritical parameters which can be changed or modified to yield essentially the same results.

### Example 1: CRISPR-Based Epigenetic Repression System

This example illustrates how to design and develop a CRISPR-based epigenetic repression system downregulating MAP2K1 and MAP2K2 expression for the treatment of neurofibromatosis type 1 (NF1).

Two key challenges remain for medical treatment of NF1 patients: 1) development of a broadly applicable therapeutic approach for all NF1 patients, and 2) the ability to load and deliver large payloads (i.e., CRISPR payloads >4.7 kb) to Schwann cells. To overcome these challenges, a novel CRISPR-based epigenetic repression system is developed to correct the underlying cause of NF1.

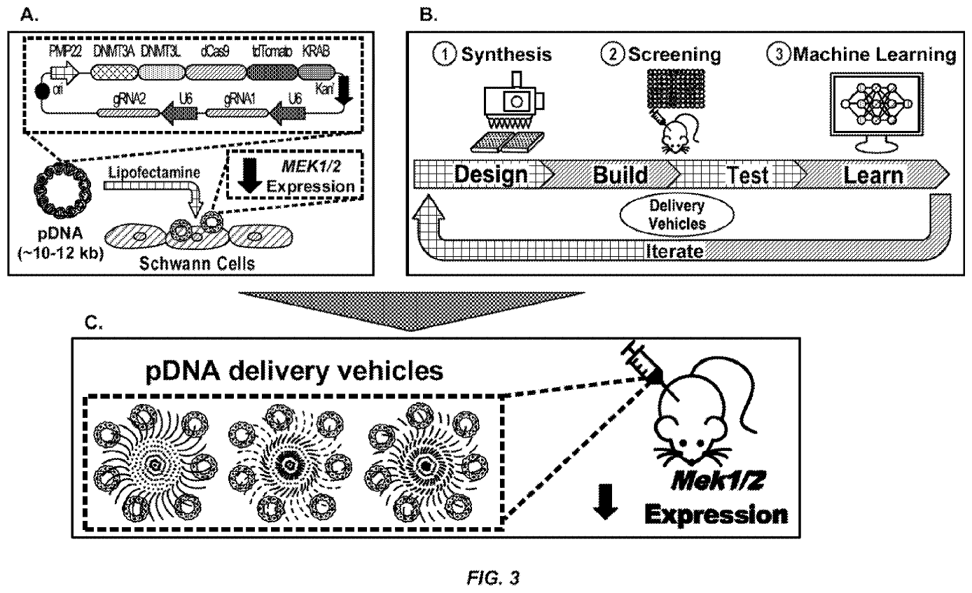

As illustrated in FIGS. 3A-3C, the system is developed through three steps: Step 1 to identify and develop gene regulatory proteins (A); Step 2 to produce delivery vehicles to deliver pDNA to Schwann cells (B); and Step 3 to test performance of the top 3 candidates in vivo (C).

The work of Step 1 is focused on creating targeted epigenetic regulatory proteins (ERPs) to be transient therapeutic payloads that cause persistent down-regulation of mitogen-activated protein kinase kinases 1 and 2 (MAP2K1/2 or MEK1/2) and delivering plasmid DNA (pDNA) encoding these ERPs to Schwann cells (SCs) in vitro and in vivo. The ERPs include a programmable DNA-binding domain such as dCas9, a repressor such as KRAB, and an effector such as DNMT3A and DNMT3L. The ERPs can be formed as a multi-protein complex or as a fusion protein.

The ERPs can be guided by one or more gRNA(s) targeting to specific gene(s) in the genomic DNA, such as the MAP2K1/2 genes. ChopChop can be used to design spCas9 guide RNAs targeting the MAP2K1 and MAP2K2 genes. All guides are chosen with the appropriate 3′ NGG PAM sequence specific for SpCas9, and with varying distances upstream of the transcriptional start site of each gene, within the promoter region. To screen the guides for the strongest repressors of each gene, vectors expressing a single guide can be co-transfected with the vector expressing the ERPs using Lipofectamine 3000 into Neuro2A cells. RNA expressed by the plasmid payload are harvested and qPCR can be used to measure the repression capabilities of the different RNA guide candidates.

As illustrated in FIG. 3A, step 1, the polynucleotide encoding all three elements of the ERPs and the polynucleotide encoding the gRNAs can be constructed in a single pDNA (~10-12 kb). Alternatively, the polynucleotide encoding all three elements of the ERPs and the polynucleotide encoding the gRNAs can be in different pDNA constructs. In addition, the polynucleotide(s) encoding the ERPs and the gRNAs can be designed to be driven by a cell-specific promoter, such as a Schwann cell-specific promoter. Therefore, the CRISPR-based epigenetic repression system will only be active in targeted cells, without impacting other cells.

Twenty guide RNA candidates (10 MAP2K1 gRNAs and 10 MAP2K2 gRNAs) were designed, developed, and screened in a preliminary study. Two guide RNAs were identified as the most promising lead candidates: MAP2K1 gRNA #7 and MAP2K2 gRNA #1. The cells expressing the ERPs and the MAP2K1 gRNA #7 reduced MAP2K1 expression by nearly 70% in comparison to the cells without a gRNA. The cells expressing the ERPs and the MAP2K2 gRNA #1 reduced MAP2K2 expression by nearly 95%. More details of the MAP2K1/2 gRNA selection are described in Example 2. These results indicate that the MAP2K1/2 gRNAs can successfully guide the ERPs to repress the MAP2K1 and MAP2K2 gene expression in neural cells. The same ERPs/gRNAs system can also be driven by a Schwann cell (SC)-specific promotor, such as the P0 or PMP22 P1 promoter, and evaluated for long-term repression capacity in Schwann cells in vitro.

As illustrated in FIG. 3B, step 2 of developing the CRISPR-based epigenetic repression system is to find the right delivery vehicle to load and deliver the ERPs/gRNAs system into the target cells such as Schwann cells in vitro and in vivo.

As illustrated in FIG. 3C, step 3 of developing the CRISPR-based epigenetic repression system is to evaluate the system delivered by the top 3 candidates in vivo. Currently, there is no safe and effective therapy specifically targeting the peripheral nervous system (PNS). By testing pDNA payload and the top three candidates for MEK1/2 downregulation in mice, this CRISPR-based epigenetic repression system can also be applied to downregulate gene expression and/or reduce certain pathway signals.

### Example 2: Development of ERP(s)/gRNA(s) Payload

This example illustrates how to design and develop the CRISPR-based epigenetic repression therapy system specifically targeting the MAP2K1 and MAP2K2 genes.

### CRISPR-Off ERP(s) Design

The CRISPR-Off ERPs include an RNA-guided programmable DNA-binding domain such as dCas9 and a repressor such as KRAB. Optionally, the CRISPR-Off ERPs can further include an effector such as DNMT3A and DNMT3L. The ERPs can be formed as a multi-protein complex or as a fusion protein.

### CRISPR-Off Guide RNA Design

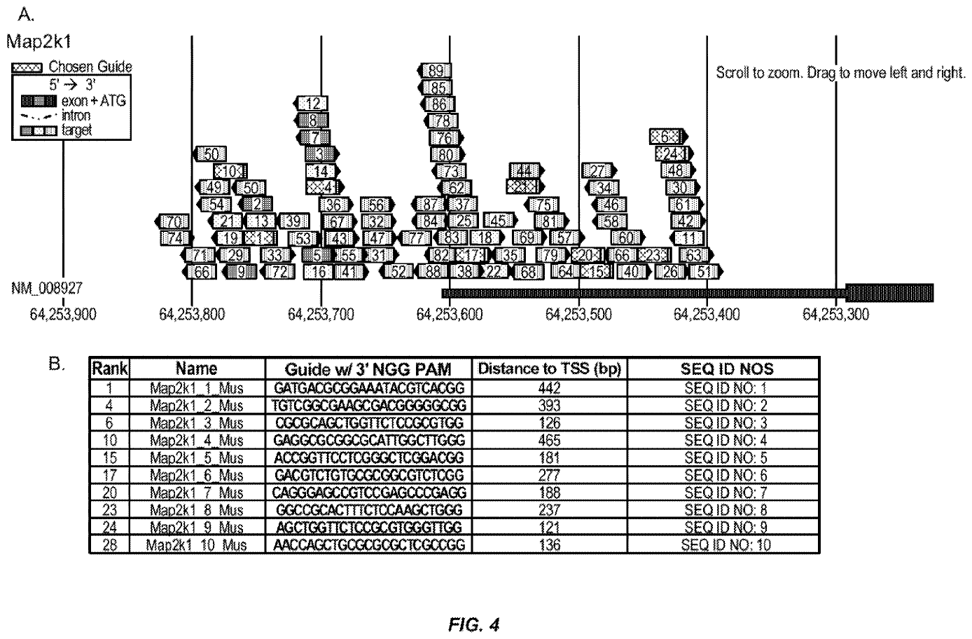

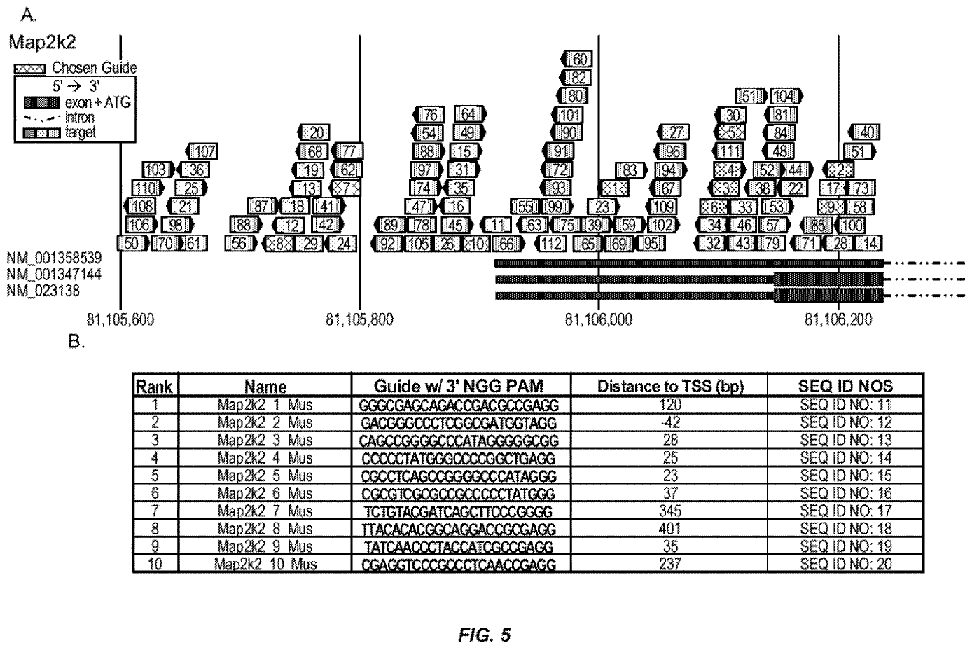

Guide RNAs targeting MAP2K1 and MAP2K2 genes were designed via the bioinformatics tool ChopChop. These gRNAs were specifically targeting 400 bp upstream of the promoters of the murine MAP2K1 and MAP2K2 genes²⁵. FIGS. 4A and 5A show the output of the ChopChop analysis of potential gRNAs of MAP2K1 and MAP2K2, respectively. Although the ChopChop algorithm ranks guides based off potential off-target affects and predicted activity levels, guide RNAs were manually selected to target different regions of the promoters to account for any variation in activity due to steric blocking by other transcription factors. Ten guide RNAs for each gene were selected, as the sequences and the distance to transcription start site (TSS) shown in FIGS. 4B and 5B, respectively.

After selecting gRNAs, each gRNA was cloned into a lenti-sgRNA puro vector (Addgene #10499). Oligonucleotides were ordered from IDT containing the 20 bp gRNA designed from ChopChop, with overhangs enabling sticky-end ligation into the vector digested with BsmBI²⁶. The lenti-sgRNA puro vector backbone was digested with BsmBI, gel purified using QIAquick Gel Extraction Kit. Oligonucleotides were phosphorylated and annealed in T4 Ligation Buffer using T4 Polynucleotide Kinase (NEB). These phosphorylated and annealed oligonucleotides were then diluted at 1:200 and ligated into the BsmBI-digested lenti-sgRNA puro vector using T4 DNA Ligase (NEB). Successful insertion of the oligonucleotides into the vector was confirmed via Sanger sequencing.

### Cell Transfection

To screen the guide RNAs for the strongest repressors of each gene, the vector expressing a single gRNA was co-transfected with a CRISPR-Off vector into Neuro2A cells. Neuro2A cells were maintained in Dulbecco's Modified Eagle Medium (DMEM) media supplemented with 10% fetal bovine serum (FBS) and Penicillin/Streptomycin at 37 C under 5% CO2. A total of 500 ng of the sgRNA expressing vector and 500 ng of the CRISPR-Off vector were transfected to 1×10⁶ Neuro2A cells at 60-70% confluency in a 12-well tissue culture plate by using Lipofectamine 3000 following the manufacturer's instructions. At day 3 post transfection, cells were harvested for quantitative PCR (qPCR) to examine repression capabilities of the different guide RNAs.

Quantitative PCR (qPCR)

Gene expression of MAP2K1 and MAP2K2 in transfected Neuro2A cells were analyzed through quantitative PCR (qPCR). Three biological replicates were tested for each sample. Total RNAs were extracted from harvested cells using Qiagen RNAeasy Micro Kit, and reverse transcribed to cDNAs using the AB high-capacity cDNA synthesis kit (ThermoFisher) following the manufacturer's instructions. The qPCR primers were designed using Primer3, with the sequences listed in Table 1. RT-qPCR was performed in triplicate using PowerUp SYBR Green Master Mix (ThermoFisher) with the CFX384 Real-Time System C1000 Touch system (Bio-Rad).

TABLE 1
qPCR Primer sequences

| Primer Name | Primer Sequence (5'-3') | SEQ ID NOS |
|---|---|---|
| MAP2Kl qPCR IF | AAGGTCTCCCACAAGCCATCTG | SEQ ID NO: 21 |
| MAP2Kl qPCR | AGTTGCACTCGTGCAGTACCTG | SEQ ID NO: 22 |
| 1R | | |
| MAP2K2 qPCR | GCCAAGCGGATTCCTGAAGACA | SEQ ID NO: 23 |
| 1F | | |
| MAP2K2 qPCR | GCGAGAGTTCACCAGGATGTTG | SEQ ID NO: 24 |
| 1R | | |
| GAPDH 1F | CATCACTGCCACCCAGAAGACTG | SEQ ID NO: |
| | | 25 |
| GAPDH 1R | ATGCCAGTGAGCTTCCCGTTCAG | SEQ ID NO: |
| | | 26 |

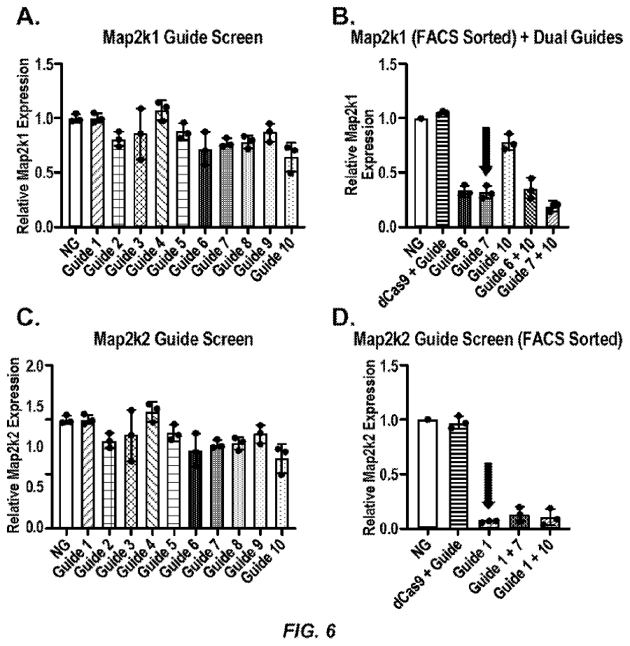

Relative target gene expression was calculated as the difference between the target gene and the GAPDH reference gene (dCq=Cq[target]−Cq[GAPDH]). Gene expression of MAP2K1 and MAP2K2 were indicated as fold change to a reference sample (CRISPR-Off without targeting gRNA (NG)) using the ddCq method, as shown in FIG. 6.

### Fluorescence-Activated Cell Sorting

To select gRNA candidates with the strongest repression capabilities on MAP2K1 and MAP2K2 gene expression in the CRISPR-Off/gRNA system, a second screening was performed through FACS (Fluorescence-activated cell sorting). FACS is a technique used to separate and isolate specific populations of cells based on their physical and fluorescent characteristics. The CRISPR-Off vector contains a blue fluorescent protein tag, cells successfully transfected with the CRISPR-Off vector will emit blue fluorescence.

The FACS sorting process involves several steps. First, the cells are suspended in a liquid stream and passed through a flow cell, where they are illuminated with a laser. As the cells pass through the flow cell, they emit fluorescent light, which is detected by a series of photomultiplier tubes. The data from the photomultiplier tubes is processed by a computer, which analyzes the amount of blue fluorescence emitted by each cell and determines which cells should be sorted. Cells that emit blue fluorescence were directly captured in 200 ul of DNA/RNA Shield (Zymo) for RNA extraction using a Qiagen RNAeasy Micro Kit.

Based on the first screening results as shown in FIGS. 6A and 6C, MAP2K1 gRNA candidates #6, #7 and #10, and MAP2K2 gRNA candidates #1, #7, #10 were selected for a second screening with FACS, with the results shown in FIGS. 6B and 6D. In addition, dual gRNAs: MAP2K1 gRNA #6 plus #10, MAP2K1 gRNA #7 plus #10, MAP2K2 gRNA #1 plus #7, and MAP2K2 gRNA #1 plus #10, were examined in case single guides did not provide adequate repression. As shown in FIGS. 6B and 6D, MAP2K1 guide #7 repressed nearly 70% MAP2K1 expression compared to the control sample without a gRNA, and MAP2K2 guide #1 repressed nearly 90% of MAP2K2 expression, indicating that MAP2K1 guide #7 and MAP2K2 guide #1 (as arrowed) are the strongest repressors in this CRISPR-Off/gRNA system. A dual guide approach yielded either similar repression capabilities, or marginally improved repressions.

### CRISPR-Off Promoter Design

The polynucleotide(s) encoding the ERPs and the gRNAs can be designed driven by a cell-specific promoter, such as a Schwann cell-specific P0 or PMP22 P1 promoter. With a Schwann cell specific promoter controlling the expression of gRNA(s), dCas9(s), repressor(s) and/or effector(s), the CRISPR-Off/gRNA system can be only selectively functioned in Schwann cells without causing side effects in other cells.

### ERP(s)/gRNA(s) Payload in One Vector

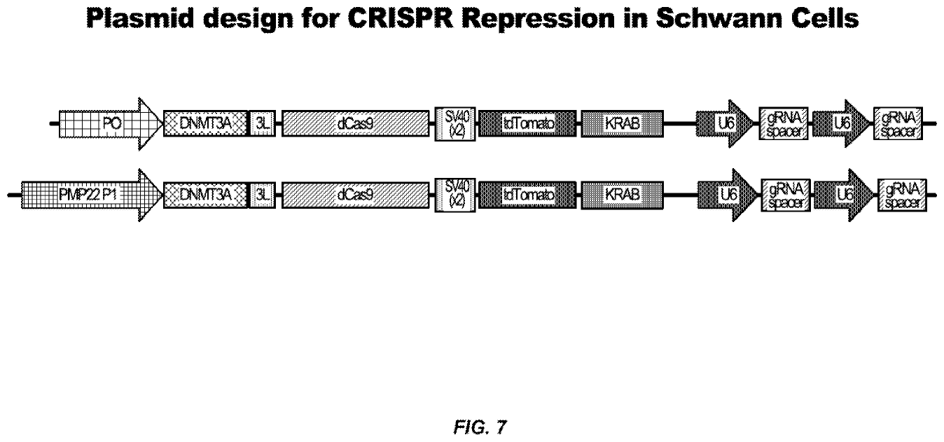

The CRISPR-based epigenetic repression system can be designed in different forms. The polynucleotide encoding the ERPs and the polynucleotide encoding the gRNAs can be in different pDNA constructs. Alternatively, the polynucleotide encoding the ERPs and the polynucleotide encoding the gRNAs can be constructed in a single pDNA. FIG. 7 illustrates a single vector that includes the entire CRISPR-Off platform and two gRNAs targeting specific genes (e.g., a MAP2K1 gRNA #7 and a MAP2K2 gRNA #1).

### Example 3: Evaluation of ERP(s)/gRNA(s) Payload

This example illustrates how to characterize the long-term repression of the MAP2K1 and MAP2K2 expression and evaluate the off-target effects of the CRISPR-Off/gRNA system.

### Long-Term Repression

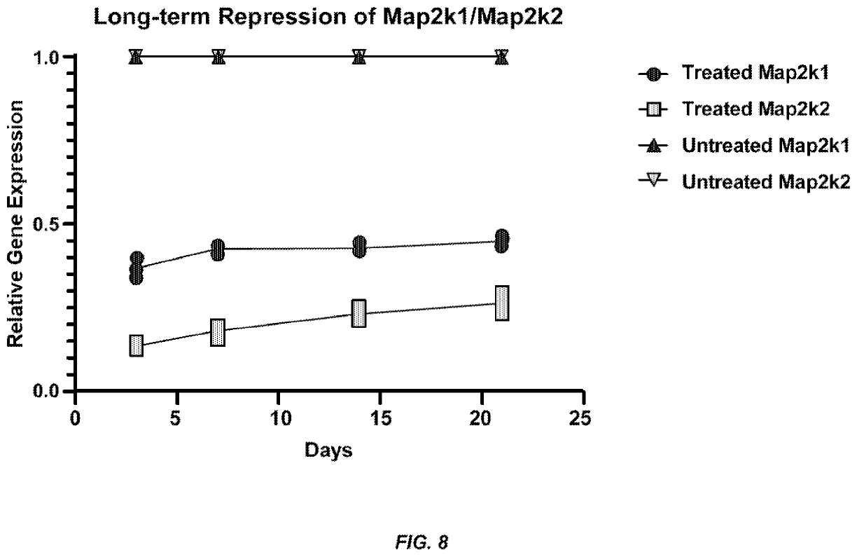

The long-term repression capabilities of both MAP2K Guide #7 and MAP2K2 Guide #1 were characterized. Neuro2A cells were lipofected with 500 ng of the CRISPR-Off vector and 500 ng of the gRNA vectors expressing MAP2K Guide #7 and MAP2K2 Guide #1. Cells successfully transfected with both CRISPER-Off and gRNA vectors were enriched through FACS at day 3 post transfection and grown for three weeks. Cells were passaged every four days, and total RNAs extracted from a portion of the cells at D3, D7, D14, and D21 post transfection for qPCR as described above. Relative target gene expression was calculated as the difference between the target gene and the GAPDH reference gene (dCq=Cq[target]-Cq[GAPDH]). Gene expression results were indicated as fold change to a reference sample (CRISPR-Off without targeting gRNA (NG)) using the ddCq method. As shown in FIG. 8, the CRISPR-Off/MAP2K1 Guide #7 system repressed about 65% of MAP2K expression at day 3 and the repression capacity was plateaued around 60% over the course of 3 weeks, and the CRISPR-Off/MAP2K2 Guide #1 system caused about 85% of MAP2K2 expression repression at day 3 and the repression capacity was gradually reduced to nearly 75% over the course of 3 weeks. This finding corroborates other studies in showing that the CRISPR-Off/gRNA system is capable of repressing genes across multiple generations²⁴.

### Off-Target Effects

This example illustrates how to evaluate the off-target effects of the CRISPR-Off/gRNA system via chromatin immunoprecipitation sequencing (Chip-seq) and RNA sequencing (RNA-seq).

Chip-seq and RNA-seq are two techniques that can be used to measure off-target effects of CRISPR gene editing. Chip-seq is a method used to identify genomic regions where a particular protein of interest is binding. In the context of CRISPR off-target effects, Chip-seq can be used to identify genomic regions where the CRISPR-Off/gRNA complex is binding (e.g., the promoter regions of the MAP2K1 and MAP2K2 genes), indicating potential off-target effects. By comparing Chip-seq data from cells (e.g., Schwann cells), that have been treated with the CRISPR-Off/gRNA system to cells that have not been treated, researchers can identify potential off-target sites. RNA-seq is a technique used to identify the types and amounts of RNA molecules present in a sample. In the context of CRISPR off-target effects, RNA-seq can be used to identify changes in gene expression that may be caused by off-target effects. By comparing RNA-seq data from cells that have been treated with the CRISPR-Off/gRNA system to cells that have not been treated, researchers can identify changes in gene expression that may be caused by off-target effects.

Both Chip-seq and RNA-seq can be used in combination to identify potential off-target effects of the CRISPR-Off/gRNA system. By identifying potential off-target sites using Chip-seq and then examining changes in gene expression using RNA-seq, researchers can gain a better understanding of the potential impact of off-target effects on cellular function.

### V. References

1. Gutmann, D. H., Wood, D. L. & Collins, F. S. Identification of the neurofibromatosis type 1 gene product. Proc Natl Acad Sci USA 88, (1991).
2. Uusitalo, E. Incidence and mortality of neurofibromatosis: a total population study in Finland. J Invest Dermatol 135, (2015).
3. Evans, D. G. Mortality in neurofibromatosis 1: in North West England: an assessment of actuarial survival in a region of the UK since 1989. Eur J Hum Genet 19, (2011).
4. Rasmussen, S. A., Yang, Q. & Friedman, J. M. Mortality in neurofibromatosis 1: an analysis using U.S. death certificates. Am J Hum Genet 68, (2001).
5. Masocco, M. & Orphanet, J. Mortality associated with neurofibromatosis type 1: a study based on Italian death certificates. Rare Dis 6, (2011).
6. Zoller, M., Rembeck, B., Akesson, H. O. & Angervall, L. Life expectancy, mortality and prognostic factors in neurofibromatosis type 1. A twelve-year follow-up of an epidemiological study in Goteborg, Sweden. Acta Derm Venereol 75, (1995).
7. Duong, T. A. Mortality associated with neurofibromatosis 1: a cohort study of 1895 patients in 1980-2006 in France. Orphanet J Rare Dis 6, (2011).
8. Messiaen, L. M. Exhaustive mutation analysis of the NF1 gene allows identification of 95% of mutations and reveals a high frequency of unusual splicing defects. Hum Mutat 15, (2000).
9. Pasmant, E. NF1 microdeletions in neurofibromatosis type 1: from genotype to phenotype. Hum Mutat 31, (2010).
10. Rojnueangnit, K. High incidence of Noonan syndrome features including short stature and pulmonic stenosis in patients carrying NF1 missense mutations affecting p. Arg1809: genotype-phenotype correlation. Hum Mutat 36, (2015).
11. Upadhyaya, M. An absence of cutaneous neurofibromas associated with a 3-bp inframe deletion in exon 17 of the NF1 gene (c.2970-2972 delAAT): evidence of a clinically significant NF1 genotype-phenotype correlation. Am J Hum Genet 80, (2007).
12. Viskochil, D. Deletions and a translocation interrupt a cloned gene at the neurofibromatosis type 1 locus. Cell 62, (1990).
13. Wallace, M. R. Type 1 neurofibromatosis gene: identification of a large transcript disrupted in three NF1 patients. Science 249, (1990).
14. DeClue, J. E. Epidermal growth factor receptor expression in neurofibromatosis type 1-related tumors and NF1 animal models. J Clin Invest 105, (2000).
15. Basu, T. N. Aberrant regulation of ras proteins in malignant tumour cells from type 1 neurofibromatosis patients. Nature 356, (1992).
16. Dasgupta, B., Yi, Y., Chen, D. Y., Weber, J. D. & Gutmann, D. H. Proteomic analysis reveals hyperactivation of the mammalian target of rapamycin pathway in neurofibromatosis 1-associated human and mouse brain tumors. Cancer Res 65, (2005).
17. Johannessen, C. M. The NF1 tumor suppressor critically regulates TSC2 and mTOR. Proc Natl Acad Sci USA 102, (2005).
18. Gutmann, D. H., Blakeley, J. O., Korf, B. R. & Packer, R. J. Optimizing biologically targeted clinical trials for neurofibromatosis. Expert Opin Investig Drugs 22, (2013).
19. Holt, S. V. et al. The MEK1/2 inhibitor, selumetinib (AZD6244; ARRY-142886), enhances anti-tumour efficacy when combined with conventional chemotherapeutic agents in human tumour xenograft models. Br. J. Cancer 106, 858-866 (2012).
20. Beert, E. Atypical neurofibromas in neurofibromatosis type 1 are premalignant tumors. Genes. Chromosomes Cancer 50, (2011).
21. Carlson-Stevermer, J. et al. CRISPRoff enables spatio-temporal control of CRISPR editing. Nat. Commun. 11, 5041 (2020).
22. Nunez, J. K. et al. Genome-wide programmable transcriptional memory by CRISPR-based epigenome editing. Cell 184, 2503-2519.e17 (2021).
23. Groner, A. C. et al. KRAB-zinc finger proteins and KAP1 can mediate long-range transcriptional repression through heterochromatin spreading. PLoS Genet. 6, e1000869 (2010).
24. Siddique, A. N. et al. Targeted methylation and gene silencing of VEGF-A in human cells by using a designed Dnmt3a-Dnmt3L single-chain fusion protein with increased DNA methylation activity. J. Mol. Biol. 425, 479-491 (2013).
25. Labun, K. et al. CHOPCHOP v3: expanding the CRISPR web toolbox beyond genome editing. Nucleic Acids Res. 47, W171-W174 (2019).
26. Stringer, B. W. et al. A reference collection of patient-derived cell line and xenograft models of proneural, classical and mesenchymal glioblastoma. Sci. Rep. 9, 4902 (2019).

It is understood that the examples and embodiments described herein are for illustrative purposes only and that various modifications or changes in light thereof will be suggested to persons skilled in the art and are to be included within the spirit and purview of this application and scope of the appended claims. All publications, patents, patent applications, and sequence accession numbers cited herein are hereby incorporated by reference in their entirety for all purposes.

### VI. Exemplary Embodiments

Exemplary embodiments provided in accordance with the presently disclosed subject matter include, but are not limited to, the claims and the following embodiments:

Embodiment 1. A recombinant polynucleotide comprising:
(a) a first nucleic acid segment encoding a fusion protein comprising an RNA-guided programmable DNA-binding domain fused to a repressor domain; and
(b) a second nucleic acid segment encoding at least one guide RNA targeting a MAP2K gene.

Embodiment 2. The polynucleotide of embodiment 1, wherein the RNA-guided programmable DNA-binding domain comprises a nuclease-deficient RNA-guided DNA endonuclease.

Embodiment 3. The polynucleotide of embodiment 2, wherein the nuclease-deficient RNA-guided DNA endonuclease is dCas9.

Embodiment 4. The polynucleotide of any embodiments of 1-3, wherein the repressor domain comprises a Krüppel-associated box (KRAB) domain, an mSin3 interaction domain (SID), a four concatenated mSin3 interaction domain (SID4X), a MAX-interacting protein 1 (MXI1), a chromo shadow domain, an EAR-repression domain (SRDX), eukaryotic release factor 1 (ERF1), eukaryotic release factor 3 (ERF3), tetracycline repressor, the lad repressor, Catharanthus roseus G-box binding factors 1 and 2, Drosophila Groucho, Tripartite motif-containing 28 (TRIM28), Nuclear receptor co-repressor 1, Nuclear receptor co-repressor 2, or any combination, fragment, or fusion thereof.

Embodiment 5. The polynucleotide of any one of embodiments 1-4, wherein the fusion protein further comprises an effector domain.

Embodiment 6. The polynucleotide of embodiment 5, wherein the effector domain comprises a DNMT3A domain, a DNMT3A with DNA Methyltransferase 3-Like protein (DNMT3A/3L) domain, a histone-lysine N-methyltransferase SUV39H1, a G9a, an enhancer of zeste homolog 2 (Ezh2), or any combination, fragment, or fusion thereof.

Embodiment 7. The polynucleotide of embodiment 5 or 6, wherein the fusion protein comprises, from N-terminus to C-terminus, the effector domain, the RNA-guided programmable DNA-binding domain, and the repressor domain.

Embodiment 8. The polynucleotide of any one of embodiments 1-7, wherein the MAP2K gene is MAP2K1 or MAP2K2.

Embodiment 9. The polynucleotide of any one of embodiments 1-8, wherein the second nucleic acid segment encodes a guide RNA targeting the MAP2K1 gene and/or a guide RNA targeting the MAP2K2 gene.

Embodiment 10. The polynucleotide of embodiment 9, wherein the guide RNA targeting the MAP2K1 gene targets a nucleic acid sequence having at least 80% identity to any one of SEQ ID NOS: 1-10.

Embodiment 11. The polynucleotide of embodiment 10, wherein the guide RNA targeting the MAP2K1 gene targets a nucleic acid sequence having at least 80% identity to SEQ ID NO: 7.

Embodiment 12. The polynucleotide of embodiment 9, wherein the guide RNA targeting the MAP2K2 gene targets a nucleic acid sequence having at least 80% identity to any one of SEQ ID NOS: 11-20.

Embodiment 13. The polynucleotide of embodiment 12, wherein the guide RNA targeting the MAP2K2 gene targets a nucleic acid sequence having at least 80% identity to SEQ ID NO: 11.

Embodiment 14. The polynucleotide of any one of embodiments 1-13, wherein the first and/or second nucleic acid segment further comprises a promoter.

Embodiment 15. The polynucleotide of embodiment 14, wherein the promoter is a Schwann cell-specific promoter.

Embodiment 16. The polynucleotide of embodiment 15, wherein the Schwann cell-specific promoter is Myelin protein zero (MPZ, P0) promoter or the peripheral myelin protein 22 (PMP22) P1 promoter.

Embodiment 17. A vector comprising the polynucleotide of any one of embodiments 1-16.

Embodiment 18. A system comprising:
(a) a first polynucleotide encoding a fusion protein comprising an RNA-guided programmable DNA-binding domain fused to a repressor domain; and
(b) a second polynucleotide encoding at least one guide RNA targeting a MAP2K gene.

Embodiment 19 A cell comprising the polynucleotide of any one of embodiments 1-16 or the vector of embodiment 17 or the system of embodiment 18.

Embodiment 20. A composition comprising the polynucleotide of any one of embodiments 1-16 or the vector of embodiment 17 or the system of embodiment 18.

Embodiment 21. A pharmaceutical composition comprising the composition of embodiment 20 and a pharmaceutically acceptable carrier.

Embodiment 22. A method for treating a RASopathy in a subject, comprising administering to the subject a therapeutically effective amount of the pharmaceutical composition of embodiment 21.

Embodiment 23. The method of embodiment 22, wherein the RASopathy is selected from the group consisting of Neurofibromatosis type 1 (NF1), Cardio-Facio-Cutaneous (CFC) syndrome, Costello syndrome (CS), Legius syndrome (LS), Capillary malformation-arteriovenous malformation (CM-AVM) syndrome, Noonan syndrome (NS), Noonan syndrome with multiple lentigines (NSML), and Noonan syndrome with loose anagen hair (NSLH).

Embodiment 24. The method of embodiment 22 or 23, wherein the RASopathy is Neurofibromatosis type 1 (NF1).

Embodiment 25. The method of any one of embodiments 22 to 24, wherein the subject is a human.

Embodiment 26. A guide RNA targeting the MAP2K1 gene having at least 80% identity to any one of SEQ ID NOS: 1-10.

Embodiment 27. The guide RNA of embodiment 26, wherein the guide RNA has at least 80% identity to SEQ ID NO: 7.

Embodiment 28. A guide RNA targeting the MAP2K2 gene having at least 80% identity to any one of SEQ ID NOS: 11-20.

Embodiment 29. The guide RNA of embodiment 28, wherein the guide RNA has at least 80% identity to SEQ ID NO: 11.

Embodiment 30. A method for downregulating a MAP2K gene expression in a subject, comprising administering to the subject a therapeutically effective amount of the pharmaceutical composition of embodiment 21.

Embodiment 31. The method of embodiment 30, wherein the subject is suffering a RASopathy disorder.

Embodiment 32. The method of embodiment 31, wherein the RASopathy disorder is Neurofibromatosis type 1 (NF1).

Embodiment 33. The method of any one of embodiments 30-32, wherein the subject is a human.

### VII. Informal Sequence Listing

| SEQ ID NO | NAMES | SEQUENCES |
|---|---|---|
| SEQ ID NO: 1 | Map2k1_1_Mus | GATGACGCGGAAATACGTCACGG |
| SEQ ID NO: 2 | Map2k1_2_Mus | TGTCGGCGAAGCGACGGGGGCGG |
| SEQ ID NO: 3 | Map2k1_3_Mus | CGCGCAGCTGGTTCTCCGCGTGG |
| SEQ ID NO: 4 | Map2k1_4_Mus | GAGGCGCGGCGCATTGGCTTGGG |
| SEQ ID NO: 5 | Map2k1_5_Mus | ACCGGTTCCTCGGGCTCGGACGG |
| SEQ ID NO: 6 | Map2k1_6_Mus | GACGTCTGTGCGCGGCGTCTCGG |
| SEQ ID NO: 7 | Map2k1_7_Mus | CAGGGAGCCGTCCGAGCCCGAGG |
| SEQ ID NO: 8 | Map2k1_8_Mus | GGCCGCACTTTCTCCAAGCTGGG |
| SEQ ID NO: 9 | Map2k1_9_Mus | AGCTGGTTCTCCGCGTGGGTTGG |
| SEQ ID NO: 10 | Map2k1_10_Mus | AACCAGCTGCGCGCGCTCGCCGG |
| SEQ ID NO: 11 | Map2k2_1_Mus | GGGCGAGCAGACCGACGCCGAGG |
| SEQ ID NO: 12 | Map2k2_2_Mus | GACGGGCCCTCGGCGATGGTAGG |
| SEQ ID NO: 13 | Map2k2_3_Mus | CAGCCGGGGCCCATAGGGGGCGG |
| SEQ ID NO: 14 | Map2k2_4_Mus | CCCCCTATGGGCCCCGGCTGAGG |
| SEQ ID NO: 15 | Map2k2_5_Mus | CGCCTCAGCCGGGGCCCATAGGG |
| SEQ ID NO: 16 | Map2k2_6_Mus | CGCGTCGCGCCGCCCCCTATGGG |
| SEQ ID NO: 17 | Map2k2_7_Mus | TCTGTACGATCAGCTTCCCGGGG |
| SEQ ID NO: 18 | Map2k2_8_Mus | TTACACACGGCAGGACCGCGAGG |
| SEQ ID NO: 19 | Map2k2_9_Mus | TATCAACCCTACCATCGCCGAGG |
| SEQ ID NO: 20 | Map2k2_10_Mus | CGAGGTCCCGCCCTCAACCGAGG |
| SEQ ID NO: 21 | MAP2Kl qPCR 1F | AAGGTCTCCCACAAGCCATCTG |
| SEQ ID NO: 22 | MAP2Kl qPCR 1R | AGTTGCACTCGTGCAGTACCTG |
| SEQ ID NO: 23 | MAP2K2 qPCR 1F | GCCAAGCGGATTCCTGAAGACA |
| SEQ ID NO: 24 | MAP2K2 qPCR 1R | GCGAGAGTTCACCAGGATGTTG |
| SEQ ID NO: 25 | GAPDH 1F | CATCACTGCCACCCAGAAGACTG |
| SEQ ID NO: 26 | GAPDH 1R | ATGCCAGTGAGCTTCCCGTTCAG |

| | |
|---|---|
| SEQ ID NO: 27 | dCAS9 (na) |

ATGGACAAGAAGTATTCTATCGGACTGGCCATCGGGACTAATAGCGTCGG
GTGGGCCGTGATCACTGACGAGTACAAGGTGCCCTCTAAGAAGTTCAAGG
TGCTCGGGAACACCGACCGGCATTCCATCAAGAAAAATCTGATCGGAGCT
CTCCTCTTTGATTCAGGGGAGACCGCTGAAGCAACCCGCCTCAAGCGGAC
TGCTAGACGGCGGTACACCAGGAGGAAGAACCGGATTTGTTACCTTCAAG
AGATATTCTCCAACGAAATGGCAAAGGTCGACGACAGCTTCTTCCATAGG
CTGGAAGAATCATTCCTCGTGGAAGAGGATAAGAAGCATGAACGGCATCC
CATCTTCGGTAATATCGTCGACGAGGTGGCCTATCACGAGAAATACCCAA
CCATCTACCATCTTCGCAAAAAGCTGGTGGACTCAACCGACAAGGCAGAC
CTCCGGCTTATCTACCTGGCCCTGGCCCACATGATCAAGTTCAGAGGCCA
CTTCCTGATCGAGGGCGACCTCAATCCTGACAATAGCGATGTGGATAAAC
TGTTCATCCAGCTGGTGCAGACTTACAACCAGCTCTTTGAAGAGAACCCC
ATCAATGCAAGCGGAGTCGATGCCAAGGCCATTCTGTCAGCCCGGCTGTC
AAAGAGCCGCAGACTTGAGAATCTTATCGCTCAGCTGCCGGGTGAAAAGA
AAAATGGACTGTTCGGGAACCTGATTGCTCTTTCACTTGGGCTGACTCCC
AATTTCAAGTCTAATTTCGACCTGGCAGAGGATGCCAAGCTGCAACTGTC
CAAGGACACCTATGATGACGATCTCGACAACCTCCTGGCCCAGATCGGTG
ACCAATACGCCGACCTTTTCCTTGCTGCTAAGAATCTTTCTGACGCCATC
CTGCTGTCTGACATTCTCCGCGTGAACACTGAAATCACCAAGGCCCCTCT
TTCAGCTTCAATGATTAAGCGGTATGATGAGCACCACCAGGACCTGACCC
TGCTTAAGGCACTCGTCCGGCAGCAGCTTCCGGAGAAGTACAAGGAAATC
TTCTTTGACCAGTCAAAGAATGGATACGCCGGCTACATCGACGGAGGTGC
CTCCCAAGAGGAATTTTATAAGTTTATCAAACCTATCCTTGAGAAGATGG
ACGGCACCGAAGAGCTCCTCGTGAAACTGAATCGGGAGGATCTGCTGCGG
AAGCAGCGCACTTTCGACAATGGGAGCATTCCCCACCAGATCCATCTTGG
GGAGCTTCACGCCATCCTTCGGCGCCAAGAGGACTTCTACCCCTTTCTTA
AGGACAACAGGGAGAAGATTGAGAAAATTCTCACTTTCCGCATCCCCTAC
TACGTGGGACCCCTCGCCAGAGGAAATAGCCGGTTTGCTTGGATGACCAG
AAAGTCAGAAGAAACTATCACTCCCTGGAACTTCGAAGAGGTGGTGGACA
AGGGAGCCAGCGCTCAGTCATTCATCGAACGGATGACTAACTTCGATAAG
AACCTCCCCAATGAGAAGGTCCTGCCGAAACATTCCCTGCTCTACGAGTA
CTTTACCGTGTACAACGAGCTGACCAAGGTGAAATATGTCACCGAAGGGA
TGAGGAAGCCCGCATTCCTGTCAGGCGAACAAAAGAAGGCAATTGTGGAC
CTTCTGTTCAAGACCAATAGAAAGGTGACCGTGAAGCAGCTGAAGGAGGA
CTATTTCAAGAAAATTGAATGCTTCGACTCTGTGGAGATTAGCGGGGTCG
AAGATCGGTTCAACGCAAGCCTGGGTACCTACCATGATCTGCTTAAGATC
ATCAAGGACAAGGATTTTCTGGACAATGAGGAGAACGAGGACATCCTTGA
GGACATTGTCCTGACTCTCACTCTGTTCGAGGACCGGGAAATGATCGAGG
AGAGGCTTAAGACCTACGCCCATCTGTTCGACGATAAAGTGATGAAGCAA
CTTAAACGGAGAAGATATACCGGATGGGGACGCCTTAGCCGCAAACTCAT
CAACGGAATCCGGGACAAACAGAGCGGAAAGACCATTCTTGATTTCCTTA
AGAGCGACGGATTCGCTAATCGCAACTTCATGCAACTTATCCATGATGAT
TCCCTGACCTTTAAGGAGGACATCCAGAAGGCCCAAGTGTCTGGACAAGG
TGACTCACTGCACGAGCATATCGCAAATCTGGCTGGTTCACCCGCTATTA
AGAAGGGTATTCTCCAGACCGTGAAAGTCGTGGACGAGCTGGTCAAGGTG
ATGGGTCGCCATAAACCAGAGAACATTGTCATCGAGATGGCCAGGGAAAA
CCAGACTACCCAGAAGGGACAGAAGAACAGCAGGGAGCGGATGAAAAGAA
TTGAGGAAGGGATTAAGGAGCTCGGGTCACAGATCCTTAAAGAGCACCCG
GTGGAAAACACCCAGCTTCAGAATGAGAAGCTCTATCTGTACTACCTTCA
AAATGGACGCGATATGTATGTGGACCAAGAGCTTGATATCAACAGGCTCT
CAGACTACGACGTGGACGCCATCGTCCCTCAGAGCTTCCTCAAAGACGAC
TCAATTGACAATAAGGTGCTGACTCGCTCAGACAAGAACCGGGGAAAGTC
AGATAACGTGCCCTCAGAGGAAGTCGTGAAAAAGATGAAGAACTATTGGC
GCCAGCTTCTGAACGCAAAGCTGATCACTCAGCGGAAGTTCGACAATCTC
ACTAAGGCTGAGAGGGGCGGACTGAGCGAACTGGACAAAGCAGGATTCAT
TAAACGGCAACTTGTGGAGACTCGGCAGATTACTAAACATGTCGCCCAAA
TCCTTGACTCACGCATGAATACCAAGTACGACGAAAACGACAAACTTATC
CGCGAGGTGAAGGTGATTACCCTGAAGTCCAAGCTGGTCAGCGATTTCAG
AAAGGACTTTCAATTCTACAAAGTGCGGGAGATCAATAACTATCATCATG
CTCATGACGCATATCTGAATGCCGTGGTGGGAACCGCCCTGATCAAGAAG
TACCCAAAGCTGGAAAGCGAGTTCGTGTACGGAGACTACAAGGTCTACGA
CGTGCGCAAGATGATTGCCAAATCTGAGCAGGAGATCGGAAAGGCCACCG
CAAAGTACTTCTTCTACAGCAACATCATGAATTTCTTCAAGACCGAAATC
ACCCTTGCAAACGGTGAGATCCGGAAGAGGCCGCTCATCGAGACTAATGG
GGAGACTGGCGAAATCGTGTGGGACAAGGGCAGAGATTTCGCTACCGTGC
GCAAAGTGCTTTCTATGCCTCAAGTGAACATCGTGAAGAAAACCGAGGTG
CAAACCGGAGGCTTTTCTAAGGAATCAATCCTCCCCAAGCGCAACTCCGA
CAAGCTCATTGCAAGGAAGAAGGATTGGGACCCTAAGAAGTACGGCGGAT
TCGATTCACCAACTGTGGCTTATTCTGTCCTGGTCGTGGCTAAGGTGGAA
AAAGGAAAGTCTAAGAAGCTCAAGAGCGTGAAGGAACTGCTGGGTATCAC
CATTATGGAGCGCAGCTCCTTCGAGAAGAACCCAATTGACTTTCTCGAAG
CCAAAGGTTACAAGGAAGTCAAGAAGGACCTTATCATCAAGCTCCCAAAG
TATAGCCTGTTCGAACTGGAGAATGGGCGGAAGCGGATGCTCGCCTCCGC
TGGCGAACTTCAGAAGGGTAATGAGCTGGCTCTCCCCTCCAAGTACGTGA
ATTTCCTCTACCTTGCAAGCCATTACGAGAAGCTGAAGGGGAGCCCCGAG
GACAACGAGCAAAAGCAACTGTTTGTGGAGCAGCATAAGCATTATCTGGA
CGAGATCATTGAGCAGATTTCCGAGTTTTCTAAACGCGTCATTCTCGCTG
ATGCCAACCTCGATAAAGTCCTTAGCGCATACAATAAGCACAGAGACAAA
CCAATTCGGGAGCAGGCTGAGAATATCATCCACCTGTTCACCCTCACCAA
TCTTGGTGCCCCTGCCGCATTCAAGTACTTCGACACCACCATCGACCGGA
AACGCTATACCTCCACCAAAGAAGTGCTGGACGCCACCCTCATCCACCAG
AGCATCACCGGACTTTACGAAACTCGGATTGACCTCTCACAGCTCGGAGG
GGAT

| | |
|---|---|
| SEQ ID NO: 28 | KRAB: (na) |

CGGACACTGGTGACCTTCAAGGATGTATTTGTGGACTTCACCAGGGAGGA
GTGGAAGCTGCTGGACACTGCTCAGCAGATCGTGTACAGAAATGTGATGC
TGGAGAACTATAAGAACCTGGTTTCCTTGGGTTATCAGCTTACTAAGCCA
GATGTGATCCTCCGGTTGGAGAAGGGAGAAGAGCCC

| | |
|---|---|
| SEQ ID NO: 29 | Dnmt3a: (na) |

ATGaaccatgaccaggaatttgaccccccaaaggtttacccacctgtgcc
agctgagaagaggaagcccatccgcgtgctgtctctctttgatgggattg
ctacagggctcctggtgctgaaggacctgggcatccaagtggaccgctac
attgcctccgaggtgtgtgaggactccatcacggtgggcatggtgcggca
ccagggaaagatcatgtacgtcggggacgtccgcagcgtcacacagaagc
atatccaggagtggggcccattcgacctggtgattggaggcagtccctgc
aatgacctctccattgtcaaccctgcccgcaagggactttatgagggtac
tggccgcctcttctttgagttctaccgcctcctgcatgatgcgcggccca
aggagggagatgatcgccccttcttctggctctttgagaatgtggtggcc
atgggcgttagtgacaagagggacatctcgcgatttcttgagtctaaccc
cgtgatgattgacgccaaagaagtgtctgctgcacacagggcccgttact
tctggggtaaccttcctggcatgaacaggcctttggcatccactgtgaat
gataagctggagctgcaagagtgtctggagcacggcagaatagccaagtt
cagcaaagtgaggaccattaccaccaggtcaaactctataaagcagggca
aagaccagcatttccccgtcttcatgaacgagaaggaggacatcctgtgg
tgcactgaaatggaaagggtgtttggcttccccgtccactacacagacgt
ctccaacatgagccgcttggcgaggcagagactgctgggccgatcgtgga
gcgtgccggtcatccgccacctcttcgctccgctgaaggaatattttgct
tgtgtg

| | |
|---|---|
| SEQ ID NO: 30 | Dnmt31: (na) |

atgggccctatggagatatacaagacagtgtctgcatggaagagacagcc
agtgcgggtactgagcctCttCagaaacatCgaCaaGgtactaaagagtt
tgggcttCttggaaagcggttctggttctgggggaggaacgctgaagtac
gtggaagatgtcacaaatgtcgtgaggagagacgtggagaaatggggccc
ctttgacctggtgtacggctcgacgcagcccctaggcagctcttgtgatc
gctgtcccggctggtacatgttccagttccaccggatcctgcagtatgcg
ctgcctcgccaggagagtcagcggcccttcttctggatattcatggacaa
tctgctgctgactgaggatgaccaagagacaactacccgcttccttcaga
cagaggctgtgaccctccaggatgtccgtggcagagactaccagaatgct
atgcgggtgtggagcaacattccagggctgaagagcaagcatgegcccct
gaccccaaaggaagaagagtatctgcaagcccaagtcagaagcaggagca
agctggacgccccgaaagttgacctcctggtgaagaactgccttctcccg
ctgagagagtacttcaagtatttttctcaaaactcacttcctctt

| | |
|---|---|
| SEQ ID NO: 31 | dCAS9 (aa) |

MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDAIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHILFTLTNLGAPAAFKYFDTTIDRKRYTSTKEVLDATLIH
QSITGLYETRIDLSQLGGD

| | |
|---|---|
| SEQ ID NO: 32 | KRAB: (aa) |

RTLVTFKDVFVDFTREEWKLLDTAQQIVYRNVMLENYKNLVSLGYQLTKP
DVILRLEKGEEP

| | |
|---|---|
| SEQ ID NO: 33 | Dnmt3a: (aa) |

MNHDQEFDPPKVYPPVPAEKRKPIRVLSLFDGIATGLLVLKDLGIQVDRY
IASEVCEDSITVGMVRHQGKIMYVGDVRSVTQKHIQEWGPFDLVIGGSPC
NDLSIVNPARKGLYEGTGRLFFEFYRLLHDARPKEGDDRPFFWLFENVVA
MGVSDKRDISRFLESNPVMIDAKEVSAAHRARYFWGNLPGMNRPLASTVN
DKLELQECLEHGRIAKFSKVRTITTRSNSIKQGKDQHFPVFMNEKEDILW
CTEMERVFGFPVHYTDVSNMSRLARQRLLGRSWSVPVIRHLFAPLKEYFA
CV

| | |
|---|---|
| SEQ ID NO: 34 | Dnmt31: (aa) |

MGPMEIYKTVSAWKRQPVRVLSLFRNIDKVLKSLGFLESGSGSGGGTLKY
VEDVTNVVRRDVEKWGPFDLVYGSTQPLGSSCDRCPGWYMFQFHRILQYA
LPRQESQRPFFWIFMDNLLLTEDDQETTTRFLQTEAVTLQDVRGRDYQNA
MRVWSNIPGLKSKHAPLTPKEEEYLQAQVRSRSKLDAPKVDLLVKNCLLP
LREYFKYFSQNSLPL

| | |
|---|---|
| SEQ ID NO: 35 | Human_DNMT3A_N612-V912 |

MNHDQEFDPPKVYPPVPAEKRKPIRVLSLFDGIATGLLVLKDLGIQVDRY
IASEVCEDSITVGMVRHQGKIMYVGDVRSVTQKHIQEWGPFDLVIGGSPC
NDLSIVNPARKGLYEGTGRLFFEFYRLLHDARPKEGDDRPFFWLFENVVA
MGVSDKRDISRFLESNPVMIDAKEVSAAHRARYFWGNLPGMNRPLASTVN
DKLELQECLEHGRIAKFSKVRTITTRSNSIKQGKDQHFPVFMNEKEDILW
CTEMERVFGFPVHYTDVSNMSRLARQRLLGRSWSVPVIRHLFAPLKEYFA
CV

| | |
|---|---|
| SEQ ID NO: 36 | Human_DNMT3L_N174-S385 |

NPLEMFETVPVWRRQPVRVLSLFEDIKKELTSLGFLESGSDPGQLKHVVD
VTDTVRKDVEEWGPFDLVYGATPPLGHTCDRPPSWYLFQFHRLLQYARPK
PGSPRPFFWMFVDNLVLNKEDLDVASRFLEMEPVTIPDVHGGSLQNAVRV
WSNIPAIRSRHWALVSEEELSLLAQNKQSSKLAAKWPTKLVKNCFLPLRE
YFKYFSTELTSS

1. A recombinant polynucleotide comprising:
(a) a first nucleic acid segment encoding a fusion protein comprising an RNA-guided programmable DNA-binding domain fused to a repressor domain; and
(b) a second nucleic acid segment encoding at least one guide RNA targeting a MAP2K gene.

2. The polynucleotide of claim 1, wherein the RNA-guided programmable DNA-binding domain comprises a nuclease-deficient RNA-guided DNA endonuclease.

3. The polynucleotide of claim 2, wherein the nuclease-deficient RNA-guided DNA endonuclease is dCas9.

4. The polynucleotide of claim 1, wherein the repressor domain comprises a Krüppel-associated box (KRAB) domain, an mSin3 interaction domain (SID), a four concatenated mSin3 interaction domain (SID4X), a MAX-interacting protein 1 (MXI1), a chromo shadow domain, an EAR-repression domain (SRDX), eukaryotic release factor 1 (ERF1), eukaryotic release factor 3 (ERF3), tetracycline repressor, the lad repressor, Catharanthus roseus G-box binding factors 1 and 2, Drosophila Groucho, Tripartite motif-containing 28 (TRIM28), Nuclear receptor co-repressor 1, Nuclear receptor co-repressor 2, or any combination, fragment, or fusion thereof.

5. The polynucleotide of claim 1, wherein the fusion protein further comprises an effector domain.

6. The polynucleotide of claim 5, wherein the effector domain comprises a DNMT3A domain, a DNMT3A with DNA Methyltransferase 3-Like protein (DNMT3A/3L) domain, a histone-lysine N-methyltransferase SUV39H1, a G9a, an enhancer of zeste homolog 2 (Ezh2), or any combination, fragment, or fusion thereof.

7. The polynucleotide of claim 5, wherein the fusion protein comprises, from N-terminus to C-terminus, the effector domain, the RNA-guided programmable DNA-binding domain, and the repressor domain.

8. (canceled)

9. The polynucleotide of claim 1, wherein the second nucleic acid segment encodes a guide RNA targeting the MAP2K1 gene and/or a guide RNA targeting the MAP2K2 gene.

10. The polynucleotide of claim 9, wherein the guide RNA targeting (i) the MAP2K1 gene targets a nucleic acid sequence having at least 80% identity to any one of SEQ ID NOS: 1-10, or (ii) the MAP2K2 gene targets a nucleic acid sequence having at least 80% identity to any one of SEQ ID NOS: 11-20.

11-13. (canceled)

14. The polynucleotide of claim 1, wherein the first and/or second nucleic acid segment further comprises a promoter.

15. The polynucleotide of claim 14, wherein the promoter is a Schwann cell-specific promoter.

16. The polynucleotide of claim 15, wherein the Schwann cell-specific promoter is Myelin protein zero (MPZ, P0) promoter or the peripheral myelin protein 22 (PMP22) P1 promoter.

17. A vector comprising the polynucleotide of claim 1.

18. A system comprising:
(a) a first polynucleotide encoding a fusion protein comprising an RNA-guided programmable DNA-binding domain fused to a repressor domain; and
(b) a second polynucleotide encoding at least one guide RNA targeting a MAP2K gene.

19. A cell comprising the polynucleotide of claim 1.

20. (canceled)

21. A pharmaceutical composition comprising the polynucleotide of claim 1 and a pharmaceutically acceptable carrier.

22. A method for treating a RASopathy in a subject, comprising administering to the subject a therapeutically effective amount of the pharmaceutical composition of claim 21.

23. The method of claim 22, wherein the RASopathy is selected from the group consisting of Neurofibromatosis type 1 (NF1), Cardio-Facio-Cutaneous (CFC) syndrome, Costello syndrome (CS), Legius syndrome (LS), Capillary malformation-arteriovenous malformation (CM-AVM) syndrome, Noonan syndrome (NS), Noonan syndrome with multiple lentigines (NSML), and Noonan syndrome with loose anagen hair (NSLH).

24-25. (canceled)

26. A guide RNA targeting (i) the MAP2K1 gene having at least 80% identity to any one of SEQ ID NOS. 1-10, or (ii) the MAP2K2 gene having at least 80% identity to any one of SEQ ID NOS. 11-20.

27-29. (canceled)

30. A method for downregulating a MAP2K gene expression in a subject, comprising administering to the subject a therapeutically effective amount of the pharmaceutical composition of claim 21.

31. The method of claim 30, wherein the subject is suffering a RASopathy disorder.

32. The method of claim 31, wherein the RASopathy disorder is Neurofibromatosis type 1 (NF1).

33. (canceled)


SEQUENCE LISTING (36 sequences)

SEQ ID NO: 1 - DNA, 23 bp, Mus musculus
  source 1..23: mol_type = other DNA
gatgacgcggaaatacgtcacgg

SEQ ID NO: 2 - DNA, 23 bp, Mus musculus
  source 1..23: mol_type = other DNA
tgtcggcgaagcgacgggggcgg

SEQ ID NO: 3 - DNA, 23 bp, Mus musculus
  source 1..23: mol_type = other DNA
cgcgcagctggttctccgcgtgg

SEQ ID NO: 4 - DNA, 23 bp, Mus musculus
  source 1..23: mol_type = other DNA
gaggcgcggcgcattggcttggg

SEQ ID NO: 5 - DNA, 23 bp, Mus musculus
  source 1..23: mol_type = other DNA
accggttcctcgggctcggacgg

SEQ ID NO: 6 - DNA, 23 bp, Mus musculus
  source 1..23: mol_type = other DNA
gacgtctgtgcgcggcgtctcgg

SEQ ID NO: 7 - DNA, 23 bp, Mus musculus
  source 1..23: mol_type = other DNA
cagggagccgtccgagcccgagg

SEQ ID NO: 8 - DNA, 23 bp, Mus musculus
  source 1..23: mol_type = other DNA
ggccgcactttctccaagctggg

SEQ ID NO: 9 - DNA, 23 bp, Mus musculus
  source 1..23: mol_type = other DNA
agctggttctccgcgtgggttgg

SEQ ID NO: 10 - DNA, 23 bp, Mus musculus
  source 1..23: mol_type = other DNA
aaccagctgcgcgcgctcgccgg

SEQ ID NO: 11 - DNA, 23 bp, Mus musculus
  source 1..23: mol_type = other DNA
gggcgagcagaccgacgccgagg

SEQ ID NO: 12 - DNA, 23 bp, Mus musculus
  source 1..23: mol_type = other DNA
gacgggccctcggcgatggtagg

SEQ ID NO: 13 - DNA, 23 bp, Mus musculus
  source 1..23: mol_type = other DNA
cagccggggcccatagggggcgg

SEQ ID NO: 14 - DNA, 23 bp, Mus musculus
  source 1..23: mol_type = other DNA
ccccctatgggccccggctgagg

SEQ ID NO: 15 - DNA, 23 bp, Mus musculus
  source 1..23: mol_type = other DNA
cgcctcagccggggcccataggg

SEQ ID NO: 16 - DNA, 23 bp, Mus musculus
  source 1..23: mol_type = other DNA
cgcgtcgcgccgccccctatggg

SEQ ID NO: 17 - DNA, 23 bp, Mus musculus
  source 1..23: mol_type = other DNA
tctgtacgatcagcttcccgggg

SEQ ID NO: 18 - DNA, 23 bp, Mus musculus
  source 1..23: mol_type = other DNA
ttacacacggcaggaccgcgagg

SEQ ID NO: 19 - DNA, 23 bp, Mus musculus
  source 1..23: mol_type = other DNA
tatcaaccctaccatcgccgagg

SEQ ID NO: 20 - DNA, 23 bp, Mus musculus
  source 1..23: mol_type = other DNA
cgaggtcccgccctcaaccgagg

SEQ ID NO: 21 - RNA, 22 nt, synthetic construct
  source 1..22: mol_type = other RNA
aaggtctcccacaagccatctg

SEQ ID NO: 22 - RNA, 22 nt, synthetic construct
  source 1..22: mol_type = other RNA
agttgcactcgtgcagtacctg

SEQ ID NO: 23 - RNA, 22 nt, synthetic construct
  source 1..22: mol_type = other RNA
gccaagcggattcctgaagaca

SEQ ID NO: 24 - RNA, 22 nt, synthetic construct
  source 1..22: mol_type = other RNA
gcgagagttcaccaggatgttg

SEQ ID NO: 25 - RNA, 23 nt, synthetic construct
  source 1..23: mol_type = other RNA
catcactgccacccagaagactg

SEQ ID NO: 26 - RNA, 23 nt, synthetic construct
  source 1..23: mol_type = other RNA
atgccagtgagcttcccgttcag

SEQ ID NO: 27 - DNA, 4104 bp, synthetic construct
  source 1..4104: mol_type = other DNA
atggacaagaagtattctatcggactggccatcgggactaatagcgtcgggtgggccgtgatcactgacgagtacaaggtgccctctaagaagttcaaggtgctcgggaacaccgaccggcattccatcaagaaaaatctgatcggagctctcctctttgattcaggggagaccgctgaagcaacccgcctcaagcggactgctagacggcggtacaccaggaggaagaaccggatttgttaccttcaagagatattctccaacgaaatggcaaaggtcgacgacagcttcttccataggctggaagaatcattcctcgtggaagaggataagaagcatgaacggcatcccatcttcggtaatatcgtcgacgaggtggcctatcacgagaaatacccaaccatctaccatcttcgcaaaaagctggtggactcaaccgacaaggcagacctccggcttatctacctggccctggcccacatgatcaagttcagaggccacttcctgatcgagggcgacctcaatcctgacaatagcgatgtggataaactgttcatccagctggtgcagacttacaaccagctctttgaagagaaccccatcaatgcaagcggagtcgatgccaaggccattctgtcagcccggctgtcaaagagccgcagacttgagaatcttatcgctcagctgccgggtgaaaagaaaaatggactgttcgggaacctgattgctctttcacttgggctgactcccaatttcaagtctaatttcgacctggcagaggatgccaagctgcaactgtccaaggacacctatgatgacgatctcgacaacctcctggcccagatcggtgaccaatacgccgaccttttccttgctgctaagaatctttctgacgccatcctgctgtctgacattctccgcgtgaacactgaaatcaccaaggcccctctttcagcttcaatgattaagcggtatgatgagcaccaccaggacctgaccctgcttaaggcactcgtccggcagcagcttccggagaagtacaaggaaatcttctttgaccagtcaaagaatggatacgccggctacatcgacggaggtgcctcccaagaggaattttataagtttatcaaacctatccttgagaagatggacggcaccgaagagctcctcgtgaaactgaatcgggaggatctgctgcggaagcagcgcactttcgacaatgggagcattccccaccagatccatcttggggagcttcacgccatccttcggcgccaagaggacttctacccctttcttaaggacaacagggagaagattgagaaaattctcactttccgcatcccctactacgtgggacccctcgccagaggaaatagccggtttgcttggatgaccagaaagtcagaagaaactatcactccctggaacttcgaagaggtggtggacaagggagccagcgctcagtcattcatcgaacggatgactaacttcgataagaacctccccaatgagaaggtcctgccgaaacattccctgctctacgagtactttaccgtgtacaacgagctgaccaaggtgaaatatgtcaccgaagggatgaggaagcccgcattcctgtcaggcgaacaaaagaaggcaattgtggaccttctgttcaagaccaatagaaaggtgaccgtgaagcagctgaaggaggactatttcaagaaaattgaatgcttcgactctgtggagattagcggggtcgaagatcggttcaacgcaagcctgggtacctaccatgatctgcttaagatcatcaaggacaaggattttctggacaatgaggagaacgaggacatccttgaggacattgtcctgactctcactctgttcgaggaccgggaaatgatcgaggagaggcttaagacctacgcccatctgttcgacgataaagtgatgaagcaacttaaacggagaagatataccggatggggacgccttagccgcaaactcatcaacggaatccgggacaaacagagcggaaagaccattcttgatttccttaagagcgacggattcgctaatcgcaacttcatgcaacttatccatgatgattccctgacctttaaggaggacatccagaaggcccaagtgtctggacaaggtgactcactgcacgagcatatcgcaaatctggctggttcacccgctattaagaagggtattctccagaccgtgaaagtcgtggacgagctggtcaaggtgatgggtcgccataaaccagagaacattgtcatcgagatggccagggaaaaccagactacccagaagggacagaagaacagcagggagcggatgaaaagaattgaggaagggattaaggagctcgggtcacagatccttaaagagcacccggtggaaaacacccagcttcagaatgagaagctctatctgtactaccttcaaaatggacgcgatatgtatgtggaccaagagcttgatatcaacaggctctcagactacgacgtggacgccatcgtccctcagagcttcctcaaagacgactcaattgacaataaggtgctgactcgctcagacaagaaccggggaaagtcagataacgtgccctcagaggaagtcgtgaaaaagatgaagaactattggcgccagcttctgaacgcaaagctgatcactcagcggaagttcgacaatctcactaaggctgagaggggcggactgagcgaactggacaaagcaggattcattaaacggcaacttgtggagactcggcagattactaaacatgtcgcccaaatccttgactcacgcatgaataccaagtacgacgaaaacgacaaacttatccgcgaggtgaaggtgattaccctgaagtccaagctggtcagcgatttcagaaaggactttcaattctacaaagtgcgggagatcaataactatcatcatgctcatgacgcatatctgaatgccgtggtgggaaccgccctgatcaagaagtacccaaagctggaaagcgagttcgtgtacggagactacaaggtctacgacgtgcgcaagatgattgccaaatctgagcaggagatcggaaaggccaccgcaaagtacttcttctacagcaacatcatgaatttcttcaagaccgaaatcacccttgcaaacggtgagatccggaagaggccgctcatcgagactaatggggagactggcgaaatcgtgtgggacaagggcagagatttcgctaccgtgcgcaaagtgctttctatgcctcaagtgaacatcgtgaagaaaaccgaggtgcaaaccggaggcttttctaaggaatcaatcctccccaagcgcaactccgacaagctcattgcaaggaagaaggattgggaccctaagaagtacggcggattcgattcaccaactgtggcttattctgtcctggtcgtggctaaggtggaaaaaggaaagtctaagaagctcaagagcgtgaaggaactgctgggtatcaccattatggagcgcagctccttcgagaagaacccaattgactttctcgaagccaaaggttacaaggaagtcaagaaggaccttatcatcaagctcccaaagtatagcctgttcgaactggagaatgggcggaagcggatgctcgcctccgctggcgaacttcagaagggtaatgagctggctctcccctccaagtacgtgaatttcctctaccttgcaagccattacgagaagctgaaggggagccccgaggacaacgagcaaaagcaactgtttgtggagcagcataagcattatctggacgagatcattgagcagatttccgagttttctaaacgcgtcattctcgctgatgccaacctcgataaagtccttagcgcatacaataagcacagagacaaaccaattcgggagcaggctgagaatatcatccacctgttcaccctcaccaatcttggtgcccctgccgcattcaagtacttcgacaccaccatcgaccggaaacgctatacctccaccaaagaagtgctggacgccaccctcatccaccagagcatcaccggactttacgaaactcggattgacctctcacagctcggaggggat

SEQ ID NO: 28 - DNA, 186 bp, synthetic construct
  source 1..186: mol_type = other DNA
cggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagccc

SEQ ID NO: 29 - DNA, 906 bp, synthetic construct
  source 1..906: mol_type = other DNA
atgaaccatgaccaggaatttgaccccccaaaggtttacccacctgtgccagctgagaagaggaagcccatccgcgtgctgtctctctttgatgggattgctacagggctcctggtgctgaaggacctgggcatccaagtggaccgctacattgcctccgaggtgtgtgaggactccatcacggtgggcatggtgcggcaccagggaaagatcatgtacgtcggggacgtccgcagcgtcacacagaagcatatccaggagtggggcccattcgacctggtgattggaggcagtccctgcaatgacctctccattgtcaaccctgcccgcaagggactttatgagggtactggccgcctcttctttgagttctaccgcctcctgcatgatgcgcggcccaaggagggagatgatcgccccttcttctggctctttgagaatgtggtggccatgggcgttagtgacaagagggacatctcgcgatttcttgagtctaaccccgtgatgattgacgccaaagaagtgtctgctgcacacagggcccgttacttctggggtaaccttcctggcatgaacaggcctttggcatccactgtgaatgataagctggagctgcaagagtgtctggagcacggcagaatagccaagttcagcaaagtgaggaccattaccaccaggtcaaactctataaagcagggcaaagaccagcatttccccgtcttcatgaacgagaaggaggacatcctgtggtgcactgaaatggaaagggtgtttggcttccccgtccactacacagacgtctccaacatgagccgcttggcgaggcagagactgctgggccgatcgtggagcgtgccggtcatccgccacctcttcgctccgctgaaggaatattttgcttgtgtg

SEQ ID NO: 30 - DNA, 645 bp, synthetic construct
  source 1..645: mol_type = other DNA
atgggccctatggagatatacaagacagtgtctgcatggaagagacagccagtgcgggtactgagcctcttcagaaacatcgacaaggtactaaagagtttgggcttcttggaaagcggttctggttctgggggaggaacgctgaagtacgtggaagatgtcacaaatgtcgtgaggagagacgtggagaaatggggcccctttgacctggtgtacggctcgacgcagcccctaggcagctcttgtgatcgctgtcccggctggtacatgttccagttccaccggatcctgcagtatgcgctgcctcgccaggagagtcagcggcccttcttctggatattcatggacaatctgctgctgactgaggatgaccaagagacaactacccgcttccttcagacagaggctgtgaccctccaggatgtccgtggcagagactaccagaatgctatgcgggtgtggagcaacattccagggctgaagagcaagcatgcgcccctgaccccaaaggaagaagagtatctgcaagcccaagtcagaagcaggagcaagctggacgccccgaaagttgacctcctggtgaagaactgccttctcccgctgagagagtacttcaagtatttttctcaaaactcacttcctctt

SEQ ID NO: 31 - protein, 1368 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 32 - protein, 62 aa, synthetic construct
RTLVTFKDVFVDFTREEWKLLDTAQQIVYRNVMLENYKNLVSLGYQLTKPDVILRLEKGEEP

SEQ ID NO: 33 - protein, 302 aa, synthetic construct
MNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACV

SEQ ID NO: 34 - protein, 215 aa, synthetic construct
MGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPL

SEQ ID NO: 35 - protein, 302 aa, Homo sapiens; identical to SEQ ID NO: 33

SEQ ID NO: 36 - protein, 212 aa, Homo sapiens
NPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFSTELTSS